(19) United States
(12) Patent Application Publication
(10) Pub. No.: US 2026/0137549 A1
(43) Pub. Date: May 21, 2026

(54) CONFIGURABLE TIME-DELAYED ORAL MANDIBLE POSITIONING DEVICE

(71) Applicant: S & V Siu Associates, LLC, Castro Valley, CA (US)

(72) Inventors: Jeremy Aidan Siu, Castro Valley, CA (US); Stanley Chan Siu, Castro Valley, CA (US)

(21) Appl. No.: 19/447,608

(22) Filed: Jan. 13, 2026

Related U.S. Application Data
(63) Continuation-in-part of application No. 18/070,939, filed on Nov. 29, 2022, now Pat. No. 12,544,254.

(51) Int. Cl. A61F 5/56

(52) U.S. Cl. CPC A61F 5/566

(57) ABSTRACT

A configurable time-delayed oral mandible displacement device includes a top plate configured for engaging maxillary teeth of a user and a bottom plate configured for engaging mandibular teeth of the user. The bottom plate is slidably coupled to the top plate for relative movement between a non-extended position and an extended position for displacing the mandible of the user during sleep. An adjustment mechanism is provided for selectably setting at least an initial position of the bottom plate relative to the top plate, within a predetermined range of initial positions, the adjustment mechanism comprising at least one element fabricated from a shape memory material (SMM).

### CROSS-REFERENCE TO RELATED APPLICATIONS

This application is a continuation-in-part of U.S. patent application Ser. No. 18/070,939, filed on Nov. 29, 2022, which claims the benefit of U.S. Provisional Application No. 63/283,919, filed on Nov. 29, 2021, the entire contents of both of these applications are incorporated herein by reference.

### FIELD OF THE INVENTION

The instant disclosure relates generally to oral appliances, and more particularly to a configurable time-delayed oral mandible displacement device for sleep apnea and snoring.

### BACKGROUND

Sleep apnea is a well-known condition that impacts millions globally. The cessation of breathing or excessive blockage of the breathing airways typically causes snoring, but the more concerning impact is that apnea impairs and interrupts normal sleep patterns. Besides the poor sleep degrading a person's productivity, apnea has the more serious impact of causing or exacerbating health conditions such as high blood pressure, heart disease, and diabetes.

Treatment or remedies for sleep apnea range from the use of continuous positive airway pressure (CPAP) machines, which force air into the sufferer's air passage, to surgical methods to widen the air passage. Disadvantages of CPAP machines include the need for a reliable source of power, noise production during operation, and being uncomfortable to wear. Surgical methods expose the sufferer to the normal risks that are associated with surgery, may require a long recovery time, and are wasteful of limited hospital resources.

Oral mandible positioning devices, which cause the sufferer to sleep with their lower jaw extended forward with respect to their upper jaw, have thus far been only moderately effective in controlling sleep apnea. These devices work by moving the lower jaw forward to enlarge the breathing airways in the nasal cavity area and thereby reduce the restriction that causes snoring and apnea. Although there are many oral mandible devices on the market, most of these devices have their configuration, or jaw forward displacement distance, preset prior to insertion into the mouth. The displacement needed to reduce snoring or apnea is typically enough to cause discomfort to the wearer of the device. The discomfort is sometimes enough to keep the user from falling asleep resulting in the user not using the device.

Some attempts have been made to provide oral mandible positioning devices that are more comfortable to wear. For example, Korean patent application publication 20210095440, by Kim Jeong Whun et al., discloses an automatic mandibular advancement device in which a power source is used to periodically provide power to a shape memory alloy spring. The spring lengthens when power is being applied to it and thereby extends a mandible positioning member relative to a maxillary support member. When power is removed the spring returns to its original shape and a return spring exerts a force to return the mandible positioning member to its original position. A controller is used to provide and remove the power at predetermined times, for example every five seconds. When the mandible positioning member is moved forward the user's lower jaw is guided to a position in which breathing is improved, but also in which the user's jaw is stressed. The stress on the user's jaw is relieved when the mandible positioning member returns to its initial position. Unfortunately, there are serious safety concerns associated with placing an electronically controlled device, containing a battery, into the user's mouth for eight or more hours each day. Additionally, some users will find the nearly continuous movement to be uncomfortable and may have difficulty falling asleep or may wake up at various times during the night.

It would therefore be beneficial to provide an improved mandible displacement device that overcomes or reduces at least some of the above-mentioned disadvantages.

### SUMMARY OF EMBODIMENTS

In accordance with at least one aspect there is provided an oral mandible displacement device that is designed to provide comfort to the user while still applying sufficient force to move the lower jaw forward and reduce or relieve the effects of airway blockage and hence, snoring and/or apnea.

In accordance with at least one aspect the device has a moldable upper and lower portion, such as for instance a moldable gel, that molds to the user's teeth and a frame with upper and lower plates that hold the gel of the moldable upper and lower portions.

In accordance with at least one aspect the device allows displacement of the wearer's lower jaw using a spring or other suitable mechanism for displacement, and a screw-type mechanism to control maximum displacement.

In accordance with at least one aspect the device addresses the major issue of initial discomfort after being inserted into the wearer's mouth by employing a shape memory material (SMM) spring to produce the force that is necessary for lower jaw displacement. Two specific and non-limiting examples include a shape memory alloy (SMA) spring or a shape memory polymer (SMP) spring. The device is configurable to provide an activation delay of desired duration such that the lower jaw is not displaced until after the wearer has fallen asleep, at which time the discomfort caused by the lower jaw displacement is not strongly felt by the wearer.

The device is configurable to provide an activation delay of at least five minutes. The device is configurable to provide a different activation delay for different users. The device is configurable to have an activation delay of between 5 minutes and 90 minutes, preferably between 10 minutes and 60 minutes, and more preferably between 15 minutes and 40 minutes.

The device is configurable to provide a desired activation delay upon being inserted into a heat space having a temperature between about 35° C. and 40° C., preferably between about 36.1° C. and 37.2° C. During use, the heat space is the mouth of a human wearer.

In accordance with at least one aspect the device is in a non-displaced (zero) or non-extended position when the wearer inserts the device into their mouth. As the device heats to near body temperature, the SMM spring begins to activate and causes the lower plate of the device to displace relative to the upper plate, such that the wearer's lower jaw is forced forward. The time delay between the initial insertion and device activation allows time for the wearer to fall asleep. In particular, the SMM spring activation is designed to occur during sleep.

In accordance with at least one aspect the device has a lead screw mechanism to adjust the maximum displacement the bottom plate can move relative to the top plate upon activation of the SMM spring. The adjustable displacement can range from 0 to 0.5 inches for a typical sized device, but for larger mouths, the range can be around 0 to 1 inch. The displacement is set by the wearer who would adjust the displacement to a level that gives the best reduction in apnea while still being comfortable.

In accordance with at least one aspect the device has two layers of heat conforming gel, one gel layer for the maxillary (upper) dentition and one gel layer for the mandibular (lower) dentition.

In accordance with at least one aspect the setting of the gel to conform to the wearer's teeth shape can be achieved by placing the device in hot water (near boiling) to soften the gel. The device is then carefully inserted into the wearer's mouth (once cooled sufficiently) and the wearer then bites down on the gel layers to form a custom mold for their teeth. As will be apparent, placing the device in hot water is expected to activate the SMM spring. The screw-type mechanism (used for setting maximum displacement during normal use) can be set to lock the lower plate in place in its non-extended condition until after the mold has set and the device is ready for use. However, for some wearers the best fit occurs when the device is extended to the wearer's desired setting before being inserted into the wearer's mouth. In this case, the screw-type mechanism is set to permit the lower plate to extend when the device is placed into hot water, and the wearer bites down on the gel layers with the device in its extended condition.

In accordance with at least one aspect the activation delay can be configured, tailored, or otherwise adjusted for the wearer by encapsulating and/or enclosing the SMM spring with various materials that can either slow or speed up the time that is required for the SMM spring to warm up to its activation temperature and activate to change shape. Encapsulating the spring in a high thermal load material, insulator, or combination of both will slow the time for the spring to reach the activation temperature as the spring relies on body heat that is absorbed from the user's mouth to warm and activate the spring. Reducing the thermal load around the SMM spring or using material that is more thermally conductive near the SMM spring will reduce the time needed for the SMM spring to activate. In this way, shorter activation times (e.g., as short as five minutes) can be provided for those wearers that tend to fall asleep quickly and longer activation times (e.g., as long as 30-60 minutes) can be provided for those wearers that tend to fall asleep more slowly.

In accordance with at least one aspect the SMM spring is a helical spring. In accordance with other aspects the SMM spring may be a flat as the design and space requirement necessitate.

In accordance with at least one aspect the device may include a ratchet and release mechanism, or another suitable locking and release mechanism, to lock the device in its extended configuration with the lower plate displace forwardly of the upper plate. After the SMM spring activates, the ratchet or other suitable locking mechanism will stop or prevent the plate from sliding back if the wearer's jaw tries to force the displacement back toward zero. The release mechanism enables the device to be reset to the zero-displacement position for the next sleep.

In accordance with at least one aspect a temperature-controlled storage device can be used to cool the oral appliance prior to use and to facilitate setting the device to its zero-displacement position. The temperature-controlled storage device is advantageous in warmer environments, in which the ambient temperature may be above the activation temperature of the SMM spring. The temperature-controlled storage device can also be used to lower the oral appliance's temperature initial temperature prior to being inserted into the wearer's mouth to lower than the ambient temperature and thereby increase the activation delay time. Further, the temperature-controlled storage device can be used to lower the oral appliance's temperature to a known initial value to ensure that the same temperature increase, and therefore the same activation delay time, will be required during each sleep before the SMM spring activates and the lower plate moves to its extended position.

In accordance with an aspect of at least one embodiment there is provided a configurable time-delayed oral mandible displacement device, comprising: a top member and a bottom member, the bottom member slidably coupled to the top member for movement between a non-extended position and an extended position; and a control mechanism coupled between the top member and the bottom member, the control mechanism comprising: an element fabricated from a shape memory material; and a heat-transfer control portion providing configurability by variation of a characteristic thereof for controllably selecting a duration of an activation time delay during which the temperature of the element fabricated from shape memory material increases to an activation temperature thereof by absorbing body heat from a human wearer after the device is inserted into the mouth of the human wearer.

In accordance with an aspect of at least one embodiment there is provided a configurable time-delayed oral mandible displacement device, comprising: a top member; a bottom member slidingly coupled to the top member for supporting a sliding movement of the bottom member relative to the top member along a displacement direction between a non-extended position and an extended position; and a control mechanism coupled between the top member and the bottom member, the control mechanism comprising a shape memory alloy portion that is at least partially contained within a heat-transfer control portion of the control mechanism, the shape memory alloy portion configured to activate upon warming to an activation temperature thereof by absorbing body heat from a human wearer of the device, wherein activation of the shape memory element includes a change of shape that provides a force for extending the bottom member from the non-extended position to the extended position, and wherein the heat-transfer control portion provides configurability by varying a characteristic thereof for controlling a duration of an activation time delay during which the temperature of the shape memory alloy portion increases to the activation temperature after the device is inserted into the mouth of the human wearer.

In accordance with an aspect of at least one embodiment there is provided a configurable time-delayed oral mandible displacement device, comprising: a top member and a bottom member slidably coupled to the top member; and an actuator disposed between the top member and the bottom member, the actuator comprising a shape memory material that is configured to provide an actuating force for slidingly extending the lower member relative to the top member upon warming of the shape memory material to an activation temperature thereof by absorbing body heat from a human user over a time period of at five minutes after being inserted into the mouth of the human user.

In accordance with an aspect of at least one embodiment there is provided a configurable time-delayed oral mandible displacement device, comprising: a top member; a bottom member slidingly coupled to the top member for supporting a sliding movement of the bottom member relative to the top member along a displacement direction between a non-extended position and an extended position; and a control mechanism disposed between the top member and the bottom member, the control mechanism comprising a shape memory alloy portion that is at least partially contained within a heat-transfer control portion of the control mechanism, the shape memory alloy portion configured to activate upon warming to an activation temperature thereof by absorbing body heat from a human user, wherein activation of the shape memory element includes a change of shape for at least one of unlocking and displacing the bottom member from the non-extended position, wherein the heat-transfer control portion provides configurability by varying a characteristic thereof for controlling a duration of an activation time delay during which the temperature of the shape memory alloy portion increases to the activation temperature after the device is inserted into the mouth of the human user.

In accordance with an aspect of at least one embodiment there is provided a configurable time-delayed oral mandible displacement device, comprising: a top plate configured for engaging maxillary teeth of a user and a bottom plate configured for engaging mandibular teeth of the user, the bottom plate slidably coupled to the top plate for relative movement between a non-extended position and an extended position for displacing the mandible of the user during sleep; and an adjustment mechanism for selectably setting at least an initial position of the bottom plate relative to the top plate, within a predetermined range of initial positions, the adjustment mechanism comprising at least one element fabricated from a shape memory material (SMM).

In accordance with an aspect of at least one embodiment there is provided a configurable time-delayed oral mandible displacement device, comprising: a top plate configured for engaging maxillary teeth of a user and a bottom plate configured for engaging mandibular teeth of the user, the bottom plate slidably coupled to the top plate for relative movement between a non-extended position and an extended position for displacing the mandible of the user during sleep; and a screw adjustment mechanism for setting an initial position of the bottom plate relative to the top plate, within a predetermined range of initial positions, and for setting a final position of the bottom plate relative to the top plate, within a predetermined range of final positions, the screw adjustment mechanism comprising: a threaded screw; a first stop member comprising a first central portion having a first threaded through-hole for engaging the threaded screw and having first and second outer portions coupled to the first central portion via a first pair of springs fabricated from a two-way shape memory material (SMM), and a second stop member comprising a second central portion having a second threaded through-hole for engaging the threaded screw and having first and second outer portions coupled to the second central portion via a second pair of springs fabricated from the two-way SMM, wherein the first pair of springs and the second pair of springs each have a remembered extended shape and a remembered non-extended shape, wherein the two-way SMM of the first set of springs is programmed such that at a first temperature the first set of springs assume the remembered extended shape and at a second temperature the first set of springs assume the remembered non-extended shape, and wherein the two-way SMM of the second set of springs is programmed such that at the first temperature the second set of springs assume the remembered non-extended shape and at the second temperature the second set of springs assume the remembered extended shape.

### BRIEF DESCRIPTION OF THE DRAWINGS

Exemplary embodiments will be described in accordance with the drawings, which are

not drawn to scale, and in which:

FIG. 1A is simplified rear isometric view of a configurable time-delayed oral mandible displacement device.

FIG. 1B is a simplified top view of the device of FIG. 1A.

FIG. 2A is a simplified cross-sectional view taken along the line A-A in FIG. 1A and showing the screw-adjustment mechanism when the device in its non-extended condition.

FIG. 2B is a simplified cross-sectional view showing the screw-adjustment mechanism of FIG. 2A when the device is in its extended condition.

FIG. 2C is a simplified front view of the device of FIG. 1A.

FIG. 3A is a simplified side view of the device of FIG. 1A in its non-extended condition.

FIG. 3B is a simplified side view of the device of FIG. 1A in its extended condition.

FIG. 4A is a simplified cross-sectional view taken along the line B-B in FIG. 1A and showing a first control mechanism in accordance with an embodiment, when the device is in its non-extended condition.

FIG. 4B is simplified a cross-sectional view showing the first control mechanism of FIG. 4A when the device is in its extended condition.

FIG. 5A is a simplified cross-sectional view taken along the line B-B in FIG. 1A and showing a second control mechanism in accordance with another embodiment, when the device is in its non-extended condition.

FIG. 5B is a simplified cross-sectional view showing the second control mechanism of FIG. 5A when the device is in its extended condition.

FIG. 5C shows enlarged detail of a portion of the simplified cross-section of FIG. 5A.

FIG. 5D shows enlarged detail of a portion of the simplified cross-section of FIG. 5B.

FIG. 6A is a simplified cross-sectional view taken along the line B-B in FIG. 1A and showing a third control mechanism in accordance with yet another embodiment, when the device is in its non-extended condition.

FIG. 6B is a simplified cross-sectional view showing the third control mechanism of FIG. 6A when the device is in its extended condition.

FIG. 6C shows enlarged detail of a portion of the simplified cross-section of FIG. 6A.

FIG. 6D shows enlarged detail of a portion of the simplified cross-section of FIG. 6B.

FIG. 6E shows further enlarged detail of the simplified cross-section of FIG. 6B including a ratchet-type structure.

FIG. 7A is a simplified cross-sectional view taken along the line B-B in FIG. 1A and showing a fourth control mechanism in accordance with still another embodiment, when the device is in its non-extended condition.

FIG. 7B is a simplified cross-sectional view showing the fourth control mechanism of FIG. 7A when the device is in its extended condition.

FIG. 7C shows enlarged detail of a portion of the simplified cross-section of FIG. 7A.

FIG. 7D shows enlarged detail of a portion of the simplified cross-section of FIG. 7B.

FIG. 8A is a simplified cross-sectional view taken along the line B-B in FIG. 1A and showing a fifth control mechanism in accordance with still another embodiment, when the device is in its non-extended condition.

FIG. 8B is a simplified cross-sectional view showing the fifth control mechanism of FIG. 8A when the device is in its extended condition.

FIG. 9A is a simplified cross-sectional view taken along the line B-B in FIG. 1A and showing a sixth control mechanism in accordance with still another embodiment, when the device is in its non-extended condition.

FIG. 9B is a simplified cross-sectional view showing the sixth control mechanism of FIG. 9A when the device is in its extended condition.

FIG. 10A is a simplified cross-sectional view taken along the line B-B in FIG. 1A and showing a seventh control mechanism in accordance with still another embodiment, when the device is in its non-extended condition.

FIG. 10B is a simplified cross-sectional view showing the seventh control mechanism of FIG. 10A when the device is in its extended condition.

FIG. 11A is a simplified diagram showing a heat transfer control portion configured to contain a shape memory material (SMM) spring and a heat transfer control gas, prior to warming the SMM spring to its activation temperature.

FIG. 11B is a simplified diagram showing the heat transfer control portion of FIG. 11A after warming the SMM spring to its activation temperature.

FIG. 11C is a simplified diagram showing a heat transfer control portion configured to contain a shape memory material (SMM) spring and a heat transfer control liquid, prior to warming the SMM spring to its activation temperature.

FIG. 11D is a simplified diagram showing the heat transfer control portion of FIG. 11C after warming the SMM spring to its activation temperature.

FIG. 11E is a simplified diagram showing a heat transfer control portion configured to contain a shape memory material (SMM) spring and a heat transfer control gel, prior to warming the SMM spring to its activation temperature.

FIG. 11F is a simplified diagram showing the heat transfer control portion of FIG. 11E after warming the SMM spring to its activation temperature.

FIG. 11G is a simplified diagram showing a heat transfer control portion configured to contain a shape memory material (SMM) spring and an insulation material, prior to warming the SMM spring to its activation temperature.

FIG. 11H is a simplified diagram showing the heat transfer control portion of FIG. 11G after warming the SMM spring to its activation temperature.

FIG. 12A is a simplified diagram showing a heat transfer control portion configured to contain a heat transfer control gas and having a central passageway for accommodating a shape memory material (SMM) spring.

FIG. 12B is a simplified diagram showing a heat transfer control portion configured to contain a heat transfer control liquid and having a central passageway for accommodating a shape memory material (SMM) spring.

FIG. 12C is a simplified diagram showing a heat transfer control portion configured to contain a heat transfer control gel and having a central passageway for accommodating a shape memory material (SMM) spring.

FIG. 12D is a simplified diagram showing a heat transfer control portion configured to contain an insulation material and having a central passageway for accommodating a shape memory material (SMM) spring.

FIG. 13 is a simplified side view showing the device of FIG. 1A with a heat transfer control portion including placement of additional thermal material.

FIG. 14A is a simplified cross-sectional view showing a configurable, time-delayed oral mandible displacement device according to an embodiment, when the device is in its non-extended condition.

FIG. 14B is simplified a cross-sectional view showing the device of FIG. 14A when the device is in its extended condition.

FIG. 15A is a partial top view showing the structure of the complementary mating structure of the device of FIGS. 14A and 14B.

FIG. 15B is a is a cross-sectional view taken along the line B-B in FIG. 15A.

FIG. 15C is a simplified isometric view showing an exemplary anchor including teeth that are formed along the side surfaces of a lower portion thereof.

FIG. 15D shows the manner in which the anchor is press-fit into the groove of complementary mating structure, prior to being press-fit.

FIG. 15E shows the manner in which the anchor is press-fit into the groove of complementary mating structure, after being press-fit.

FIG. 16A is a is a simplified cross-sectional view showing a configurable, time-delayed oral mandible displacement device according to an embodiment, when the device is in its non-extended condition.

FIG. 16B is simplified a cross-sectional view showing the device of FIG. 16A when the device is in its extended condition.

FIG. 17A is a partial top view showing the structure of the complementary mating structure of the device of FIGS. 16A and 16B.

FIG. 17B is a is a cross-sectional view taken along the line B-B in FIG. 17A.

FIG. 18A is a simplified cross-sectional view of a configurable, time-delayed oral mandible displacement device according to an embodiment and showing a screw-adjustment mechanism for setting an initial position of the bottom plate relative to the top plate, when the device in its non-extended condition and the top and bottom plates are aligned.

FIG. 18B is a simplified cross-sectional view showing the device of FIG. 18A in its extended condition.

FIG. 19A is a simplified cross-sectional view of the device of FIG. 18A when the device in its non-extended condition and the initial position of the bottom plate protrudes beyond the top plate.

FIG. 19B is a simplified cross-sectional view showing the device of FIG. 19A in its extended condition.

FIG. 20A is a is a simplified cross-sectional view of a configurable, time-delayed oral mandible displacement device according to an embodiment and showing a screw-adjustment mechanism for setting both an initial position and a final position of the bottom plate relative to the top plate, when the device in its non-extended condition and the top and bottom plates are aligned.

FIG. 20B is a simplified cross-sectional view showing the device of FIG. 20A in its extended condition.

FIG. 21A is a simplified cross-sectional view of the device of FIG. 20A when the device in its non-extended condition and the initial position of the bottom plate protrudes beyond the top plate.

FIG. 21B is a simplified cross-sectional view showing the device of FIG. 21A is in its extended condition.

FIG. 22A is a simplified diagram showing an expandable adjustment block according to an embodiment, when in a non-adjustable condition.

FIG. 22B is a simplified diagram showing the expandable adjustment block of FIG. 22A, when in an adjustable condition.

FIG. 22C is an isometric view of the expandable adjustment block of FIG. 22A, when in an adjustable condition.

FIG. 23 is a simplified diagram showing a device according to an embodiment with shape memory material hinge elements for maintaining close fit to the wearer's teeth.

FIG. 24 is a simplified diagram showing a device according to an embodiment with spring elements for maintaining close fit to the wearer's teeth.

FIG. 25 is a simplified diagram showing an implementation for preventing movement of the bottom plate away from its desired final position relative to the top plate.

FIG. 26 is a simplified diagram showing another implementation for preventing movement of the bottom plate away from its desired final position relative to the top plate, prior to activation.

FIG. 27 is a simplified diagram showing the implementation of FIG. 26, after activation.

FIG. 28 is a simplified diagram showing another implementation for preventing movement of the bottom plate away from its desired final position relative to the top plate, prior to activation.

FIG. 29 is a simplified diagram showing the implementation of FIG. 28, after activation.

FIG. 30 is a simplified diagram showing another implementation for preventing movement of the bottom plate away from its desired final position relative to the top plate, prior to activation.

FIG. 31 is a simplified diagram showing the implementation of FIG. 29, after activation.

FIG. 32 is a simplified diagram showing a releasable locking mechanism according to an embodiment, in a locked condition

FIG. 33 is a simplified diagram showing the locking mechanism of FIG. 32, in an unlocked condition.

FIG. 34 is a simplified diagram showing a releasable locking mechanism according to another embodiment, in a locked condition

FIG. 35 is a simplified diagram showing the locking mechanism of FIG. 34, in an unlocked condition.

FIG. 36 is a simplified diagram showing an implementation of a replaceable impression tray according to an embodiment.

### DETAILED DESCRIPTION OF THE DRAWINGS

While the present teachings are described in conjunction with various

embodiments and examples, it is not intended that the present teachings be limited to such embodiments. On the contrary, the present teachings encompass various alternatives and equivalents, as will be appreciated by those of skill in the art. All statements herein reciting principles, aspects, and embodiments of this disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

### 1. Definitions

The terms “top,” “bottom,” “upper,” “lower,” “front,” “rear” and similar relative terms are used only for convenience when describing the various embodiments as they are oriented in the accompanying drawings.

“Shape memory material”—includes shape memory alloys, shape memory polymers, shape changing polymers and the like. A one-way shape memory material is one that can be deformed when cooled and returns to its pre-deformed (“remembered”) shape when warmed. When the one-way shape memory material cools again, it will retain its shape until deformed again. Cooling from high temperature to low temperature does not cause a one-way shape memory material to return to another “remembered” shape. A two-way shape memory material “remembers” two different shapes: one at low temperature and one at high temperature. When cooled the material returns to its “remembered” low temperature shape without requiring deformation and when heated the material returns to its “remembered” high temperature shape. Shape memory materials also includes three-way shape memory materials.

### 2. Exemplary Device

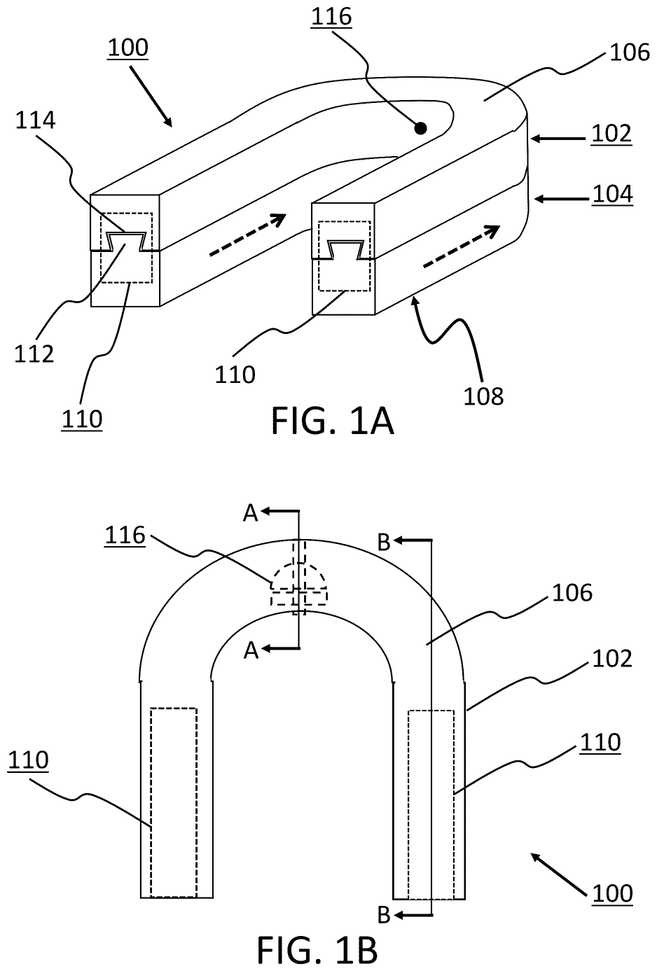

Referring to FIG. 1A, shown is a simplified rear isometric view of an exemplary, configurable time-delayed oral mandible displacement device 100. The device 100 includes a top plate 102, also referred to equivalently as a top member 102. The device 100 further includes a bottom plate 104, also referred to equivalently as a bottom member 104. In the embodiment that is illustrated in FIG. 1A, both the top plate 102 and the bottom plate 104 are substantially U-shaped to match the dentition of a wearer of the device, also referred to equivalently as a user or as a human user. The top plate 102 and the bottom plate 104 may be fabricated using any material that is generally suitable for use in oral appliance applications, such as for instance medical grade PVC or Polyethylene, PEEK, Polycarbonate, Polypropylene and Polyurethane, etc.

The top plate 102 has an upper surface 106 for receiving the maxillary dentition of the wearer and the bottom plate 104 has a lower surface 108 for receiving the mandibular dentition of the wearer. The upper surface 106 and the lower surface 108 are each provided with a (not illustrated in FIG. 1A) layer of moldable gel. During a fitting procedure, the moldable gel is softened by heating and the wearer bites down on the gel layers to create an impression therein matching their unique dental structure. After hardening, the impressions formed in the gel layers are precisely matched to the wearer's dental structure, forming a close fit therebetween, which acts to secure the device 100 within the wearer's mouth. The moldable gel is any moldable gel that is suitable for use in creating dentition impressions in oral appliances, such as for instance ethylene-vinyl acetate, commonly known as EVA.

Referring still to FIG. 1A, the top plate 102 and the bottom plate 104 are slidingly coupled together for supporting a sliding movement of the bottom plate 104 relative to the top plate 102 along a displacement direction, which is indicated by the dashed arrows. FIG. 1A shows the device 100 in a configuration that is intended for being inserted into the mouth of the wearer, in which the bottom plate 104 is in a non-extended position relative to the top plate 102. During use, after being inserted into the wearer's mouth, the device 100 is activated and the bottom plate 104 extends along the displacement direction. The top plate 102 is held in place by the wearer's mandibular dentition, which does not move relative to the rest of the wearer's skull. Thus, movement of the bottom plate 104 relative to the top plate 102 along the displacement direction causes the wearer's lower jaw to also move forward and into a position in which the breathing airways in the wearer's nasal cavity area become enlarged.

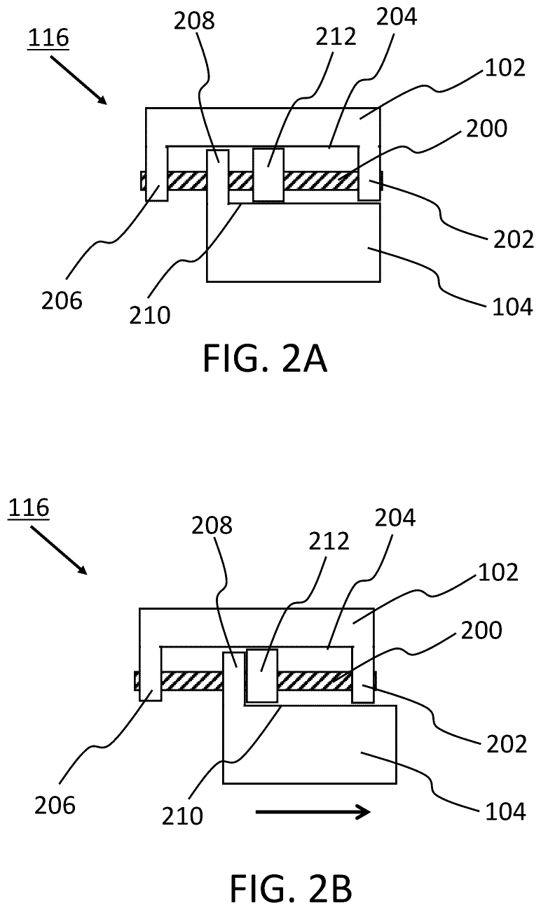

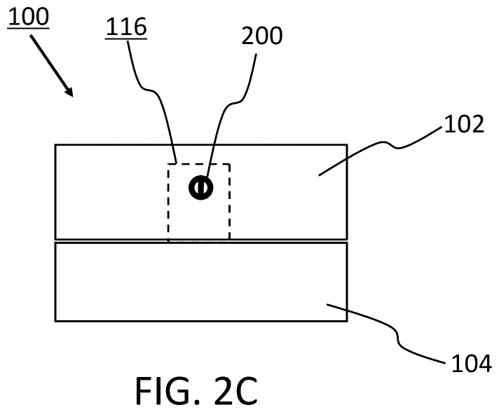

The device 100 shown in FIG. 1A further includes a coupling and actuation portion that is shown generally within the dashed line box 110, which includes a control mechanism capable of being configured to provide different activation times. In this specific and non-limiting example, each arm of the generally U-shaped bottom plate 104 includes a rail 112 extending along the displacement direction and each arm of the generally U-shaped top plate 102 includes a groove 114 extending along the displacement direction. Each rail 112 is configured to slide within a complementary shaped groove 114, thereby permitting relative motion between the top plate 102 and the bottom plate 104 along only one direction. In FIG. 1A, each rail 112 and each groove 114 has a shape that is generally an isosceles trapezoid in a cross-section taken in a plane perpendicular to the displacement direction. Of course, other configurations of the rails 112 and grooves 114 may be envisaged, and other suitable systems for slidingly coupling the top plate 102 and the bottom plate 104 may be substituted for the illustrated rails 112 and grooves 114. The maximum displacement of the bottom plate 104 relative to the top plate 102 is limited using an adjustment mechanism, such as for instance a screw-adjustment mechanism shown partially at 116 and described in greater detail below with reference to FIGS. 2A-2C.

FIG. 1B is a simplified top view of the device 100 in its non-extended condition. The dashed line box 110 illustrates the general location of the coupling and actuation portion of FIG. 1A, which is arranged along the facing surfaces of the top plate 102 and of the bottom plate 104. The screw-adjustment mechanism 116, which is disposed between the top plate 102 and the bottom plate 104, is shown using dashed lines in FIG. 1B.

Referring now to FIG. 2A and FIG. 2B, shown are cross-sectional views taken along the line A-A of FIG. 1B, showing the screw-adjustment mechanism 116 when the device 100 is in its non-extended condition and when the device 100 is in its extended condition, respectively. The screw-adjustment mechanism 116 includes a threaded screw 200 that is retained within respective openings, at least some of which are also threaded, formed through a first post 202 extending from a lower surface 204 of the top plate 102, a second post 206 extending from the lower surface 204 of the top plate 102, and a third post 208 extending from the upper surface 210 of the bottom plate 104. An adjustment block 212 moves along the length of the threaded screw 200 when the threaded screw 200 is turned. As shown in FIG. 2B, when the bottom plate 104 is displaced along the displacement direction indicated by the arrow, the adjustment block 212 and the third post 208 extending from the upper surface 210 of the bottom plate 104 come into contact one with the other and thereby prevent further displacement of the bottom plate 104 along the displacement direction.

Referring now to FIG. 2C, shown is a simplified front view of the device 100. Certain features have been omitted in FIG. 2C for improved clarity, such as for instance vent slots that may be provided to improve airflow. The end of the threaded screw 200 of the screw-adjustment mechanism 116 is preferably accessible and provided with a feature such as a slot-shaped recess or an X-shaped recess for allowing the wearer to adjust the screw-adjustment mechanism 116 using e.g., a slot screwdriver or a Philips screwdriver, etc. The wearer may desire to adjust the screw-adjustment mechanism 116 for comfort, including an initial adjustment when the device is new and from time-to-time thereafter if the device starts to become uncomfortable to wear.

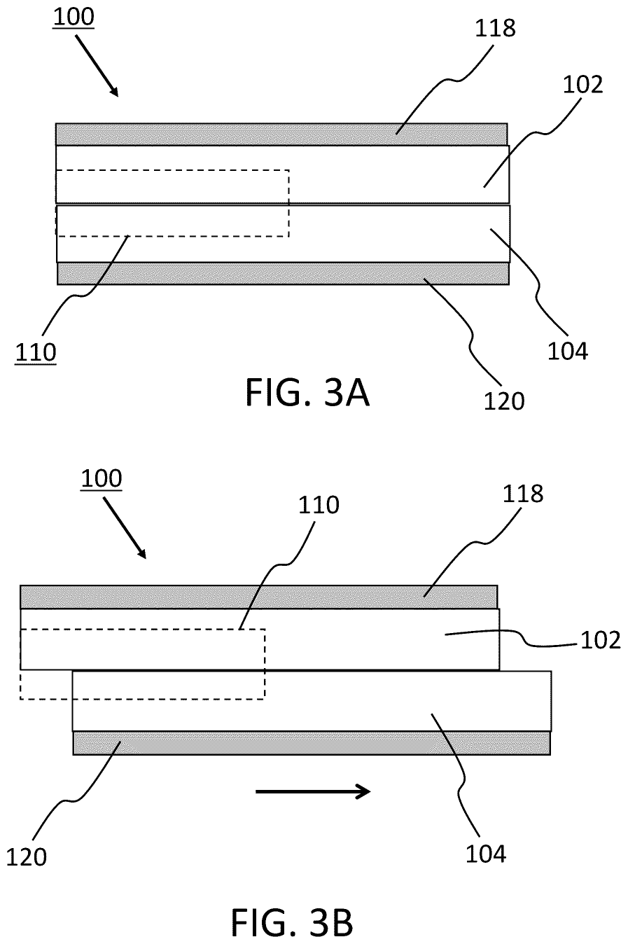

FIG. 3A is a simplified side view showing the device 100 in its non-extended condition. The front surface of the top plate 102 and the front surface of the bottom plate 104 are generally aligned in the non-extended condition, which minimizes the size of the device to facilitate insertion into the mouth of the wearer. Optionally, the bottom plate 104 extends slightly forward of the top plate 102 along the displacement direction when the device is in its non-extended condition, which reduces the distance of travel of the bottom plate when the device 100 is activated and may be less disturbing to some wearers. FIG. 3B is a simplified side view showing the device 100 in its extended condition. As discussed with reference to FIGS. 2A and 2B, the maximum displacement of the bottom plate 104 relative to the top plate 102 is limited using a suitable adjustment mechanism, such as for instance screw-adjustment mechanism 116 (not illustrated in FIG. 3B).

FIG. 3A. and FIG. 3B also show a first layer of moldable gel 118 arranged along the upper surface 106 of top plate 102 and a second layer of moldable gel 120 arranged along the lower surface 108 of the bottom plate 104. As discussed above with reference to FIG. 1A, the first layer of moldable gel 118 and the second layer of moldable gel 120 are heated during an initial fitting step, and the wearer subsequently bites down on the softened gel to create impressions of the maxillary and mandibular dentition, respectively. For better clarity, the first layer of moldable gel 118 and the second layer of moldable gel 120 have been omitted in the remaining figures.

### 3. Operation

Device 100 is a configurable time-delayed oral mandible displacement device for sleep apnea and snoring. The coupling and actuation portion indicated generally at 110, which includes a control mechanism for providing a desired activation delay time, has already been described above in general terms. Structure, such as for instance rails 112 and grooves 114, is provided for slidingly coupling the top plate 102 and the bottom plate 104. In addition, the control mechanism of the coupling and actuation portion 110 includes at least an element for storing potential energy for providing the force that is required to displace the bottom plate 104 relative to the top plate 102, a specific and non-limiting example being a spring. Further, the control mechanism is configurable to allow the selection of a desired activation delay between the time the device 100 is inserted into the wearer's mouth in its non-extended condition and the time the bottom plate 104 moves along the displacement direction relative to the top plate 102 such that the device 100 is in its extended condition. Various control mechanisms, which are suitable for providing the described functionality, are discussed in the following sections and are merely some specific and non-limiting examples. Other control mechanisms may be envisaged without departing from the scope of the invention.

### 4. First Control Mechanism

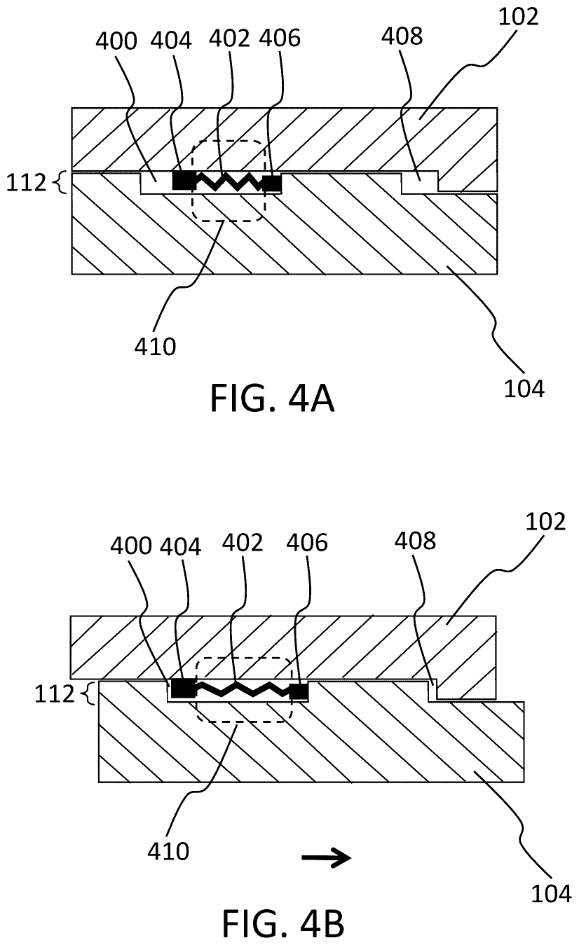

Referring now to FIG. 4A, shown is a cross-sectional view of device 100 taken along line B-B in FIG. 1B, with a first control mechanism in accordance with an embodiment, and when the device 100 is in its non-extended condition. As shown in FIG. 4A, the rail 112 extending from the upper surface of bottom plate 104 is non-continuous and includes a gap 400. Disposed within gap 400 is an element for storing potential energy in the form of a spring fabricated from a shape memory material (SMM), i.e., SMM spring 402. SMM spring 402 may be fabricated from a shape memory alloy, a shape memory polymer, a shape change polymer etc., and the material may be a one-way SMM, a two-way SMM or a three-way SMM, etc. Nitinol, a binary alloy of titanium with 45% to 50% nickel is one example of a SMM that can have an activation temperature in the body temperature range and provide sufficient spring energy for this device. Nitinol can be a one-way or two-way SMM. Polymers such as IPDI-PCL-BDO, POSS-PDLLA-co-CL, and MM3520 from SMP Technologies are polymers with SMM characteristics, such as body temperature activation temperature, that may be used in this device.

Referring still to FIG. 4A, the SMM spring 402 is attached to the top plate 102 via attachment point 404 and is attached to the bottom plate 104 via attachment point 406. A gap 408 is provided between the top plate 102 and the bottom plate 104 to allow room for the bottom plate 104 to slide along the displacement direction relative to the top plate 102. During use, the device 100 is cooled below an activation temperature of the SMM spring 402 and is subsequently inserted into the mouth of a wearer in its non-extended condition shown in FIG. 4A. Body heat from the wearer, which on average is between about 36.1° C. and 37.2° C., warms the material of the device 100, including the SMM spring 402. When the SMM spring 402 warms to its activation temperature, it “remembers” its previously programmed high temperature shape and activates, thereby causing the distance between the attachment points 404 and 406 to increase as the SMM spring lengthens in this specific example. As a result, the bottom plate 104 is displaced forwardly with respect to the top plate 102, causing the device to be in its extended condition as shown in FIG. 4B, in which gaps 400 and 408 are both reduced in size.

Now referring to FIG. 4A and FIG. 4B, a heat transfer control portion is indicated generally at 410 using a dashed-line rounded rectangle. The heat transfer control portion 410 is configurable to affect the rate at which body heat from the wearer's mouth is absorbed by the SMM spring 402, and thereby allowing the device 100 to be configured for different activation times for different wearers. For instance, the heat transfer control portion 410 is configured to slow the rate of heat transfer if the wearer takes longer to fall asleep, or the heat transfer control portion 410 is configured to maximize the rate of heat transfer if the wearer takes less time to fall asleep. Various modifications to the heat transfer control portion 410 may be selected to tailor the rate of heat transfer to produce an activation delay time that is suitable for a wide range of wearer's requirements.

The heat transfer control portion 410 may include a separate enclosure that partially or completely surrounds or contains the SMM spring 402. The material used to fabricate such a separate enclosure and/or a material thickness of at least a portion of the separate enclosure and/or a medium contained within the separate enclosure may be varied to produce a desired heat transfer rate and accordingly to provide a desired activation delay. In some embodiments, the separate enclosure may be adjacent to the SMM spring 402 along only one side thereof, or it may partially or completely enclose the SMM spring 402.

In some embodiments the separate enclosure may be fabricated from a metal to provide higher thermal conduction. Some non-limiting examples of suitable metals include various titanium alloys including Grade 5 Titanium alloy (Ti-6Al-4V) which is used for implants and dental appliances. The metal may be made to be porous to adjust the effective thermal conductivity. Alternatively, a ceramic, porous ceramic, insulating polymer such as Styrofoam®, or other material may be used to decrease the thermal conductivity or thermal load for longer activation delay of the SMM spring 402. Some non-limiting examples of ceramics are alumina and zirconia. The material thickness of different portions of the wall of the separate enclosure may also be different.

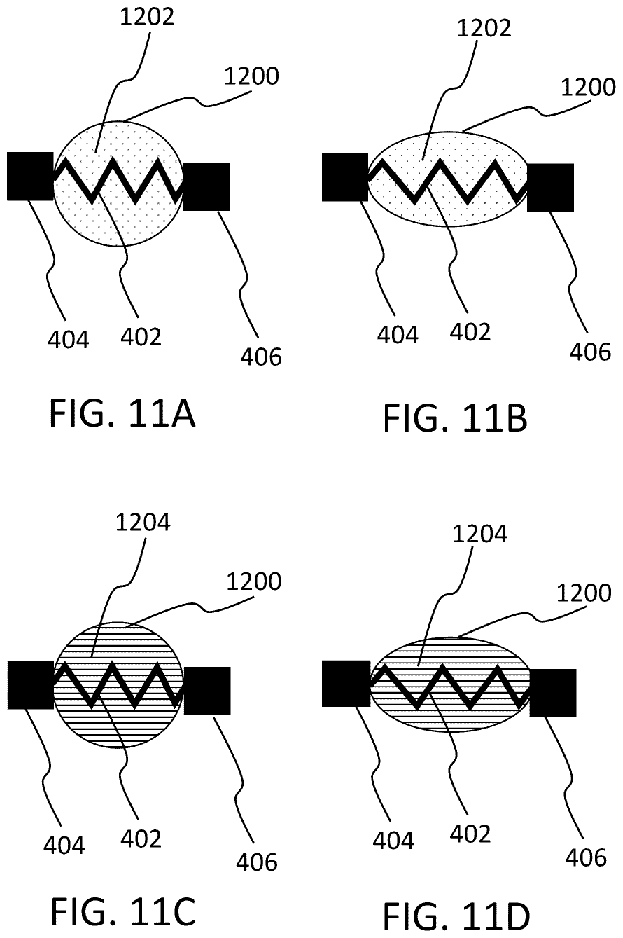

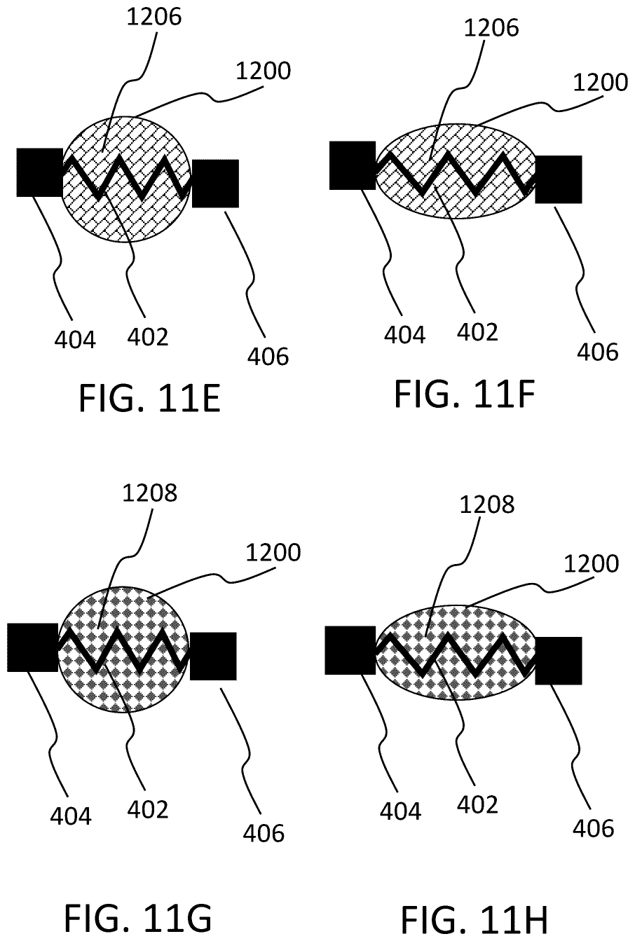

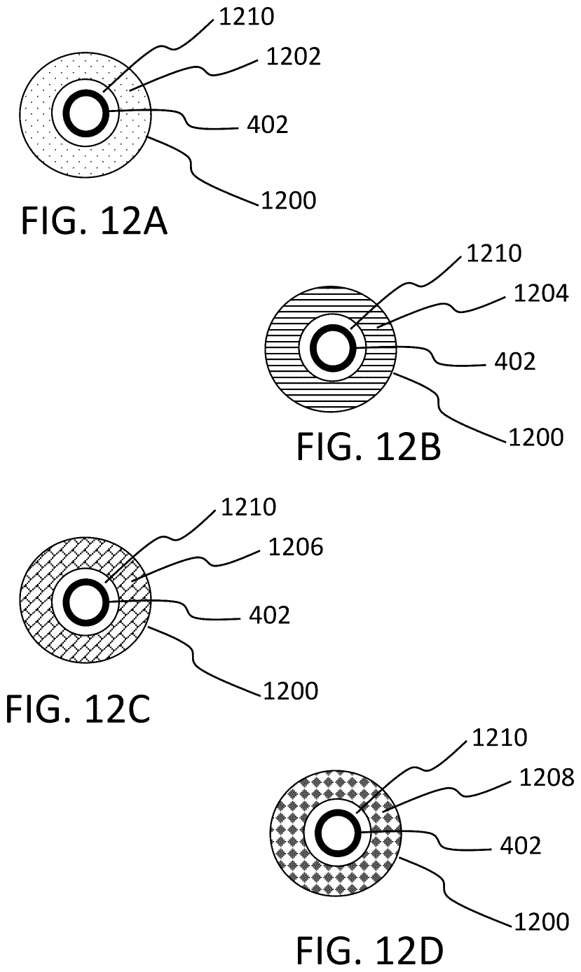

In some embodiments the separate enclosure may be formed using a flexible, liquid-tight polymer such as polyvinylidene chloride, i.e., a plastic film/membrane. In such embodiments, the enclosure preferably contains a material that is selected to affect the rate at which the wearer's body heat is transferred to the SMM spring 402. The material may be any suitable gas, liquid, gel, or a solid insulating material, such as for instance Styrofoam® beads. The enclosure is flexible and changes shape as the SMM spring 402 expands or contracts along the displacement direction. Various configurations are discussed in more detail with reference to FIGS. 11A-11H and FIGS. 12A-12D.

In some embodiments the material or material thickness used in the top plate 102 and/or bottom plate 104 is part of the heat transfer control portion 410 and is selected to control the rate of heat transfer. Optionally, portions of the top plate 102 and/or bottom plate 104 proximate the SMM spring 402 include structures for receiving inserts fabricated from a material that can be selected to provide a desired heat transfer rate.

After the wearer removes the device 100 from their mouth, it must be reset back to its non-extended condition before the next sleep. The SMM spring 402 must be cooled below its activation temperature before it can be deformed, and the device returned its non-extended condition. When the SMM spring 402 is fabricated using a one-way shape memory material, an external force must be applied to slide the bottom plate 104 back to the non-extended condition. When the SMM spring 402 is fabricated from a two-way shape memory material, the SMM spring 402 will “remember” its low temperature shape upon sufficient cooling and the bottom plate 104 will slide back to the non-extended condition absent application of an external force, or with only a relatively small external force. In cooler climates the ambient temperature may be sufficiently below the activation temperature to cool the SMM spring 402 such that the device 100 can be reset to its non-extended condition without requiring supplemental cooling. In warmer climates and/or for devices that are configured for very slow heat transfer and long activation delay times, a temperature-controlled storage device may be required to cool the device 100 sufficiently and/or more quickly than is possible under ambient conditions. The temperature-controlled storage device may include a chilled enclosure for storing the device 100, and optionally the enclosure contains a chilled liquid which may be a cleaning/sanitizing liquid for the device 100.

In sum, the heat transfer control portion 410 may include portions of the top plate 102, portions of the bottom plate 104, and/or other separate components that may be incorporated into device 100. By varying the shape, thickness, composition, arrangement etc. of the various portions and components as described above, it is possible to place materials between the SMM spring 402 and the wearer to control the rate at which body heat from the wearer is transferred to and subsequently absorbed by the SMM spring 402. Since body temperature is substantially constant from one wearer to another, and assuming a standard initial temperature of the device 100 as obtained e.g., using a temperature-controlled storage device, it is possible to select an appropriate configuration of the heat transfer control portion 410 to provide activation delay times over a wide range of values. In general, a minimum activation delay time for device 100 is 5 minutes. Wearer-specific activation delay times may be in the range between 5 minutes and 90 minutes, preferably between 10 minutes and 60 minutes, and more preferably between 15 minutes and 40 minutes.

### 5. Second Control Mechanism

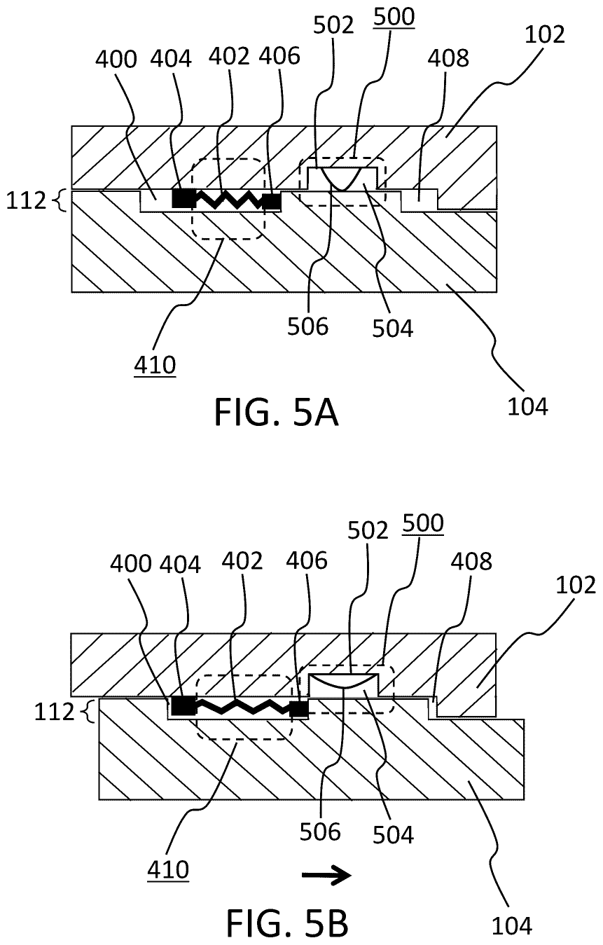

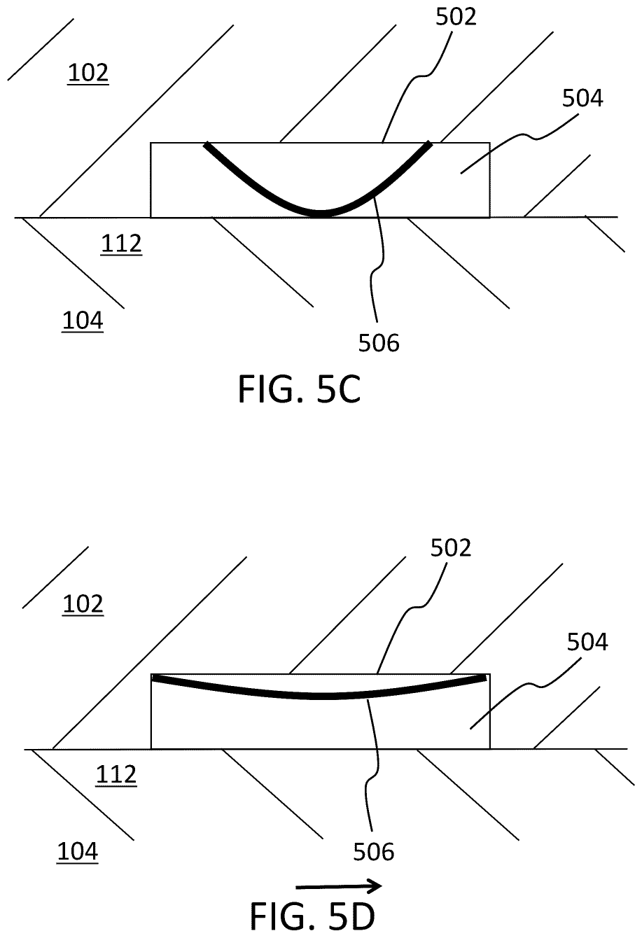

Referring now to FIG. 5A, shown is a cross-sectional view of device 100 taken along line B-B in FIG. 1B, with a second control mechanism in accordance with an embodiment, and when the device 100 is in its non-extended condition. The elements of the second control mechanism include the same elements that have already been described with reference to FIG. 4A and FIG. 4B, and additionally include a locking mechanism shown generally at 500. During use, the locking mechanism 500 can be reset when the device 100 is returned to its non-extended condition after being worn by the wearer. When reset, i.e., in an engaged condition, the locking mechanism 500 locks the device 100 in its non-extended condition.

In this specific and non-limiting example, the locking mechanism 500 is disposed within a recess 502 that is formed in the groove 114 along the lower surface of the top plate 102. The recess 502 provides a space 504 to accommodate a lock-element 506 formed from a SMM, such as for instance a generally strip-shaped piece of SMM that is more strongly curved when it is below its activation temperature than when it is above its activation temperature. The lock-element 506 may be formed using the same material that is used to form SMM spring 402, or a different suitable shape memory material having an activation temperature close to average human body temperature may be used instead. Optionally, the lock-element 506 is associated with a not illustrated heat-transfer control portion to provide an activation delay prior to activating, similar to the function of the already-described heat transfer portion 410. However, since the locking mechanism 500 in the current embodiment is intended primarily to hold the device 100 in its non-extended condition only until after the device 100 has been inserted into the wearer's mouth, in most cases it is not necessary to provide an activation delay prior to activation of the lock-element 506, or at least it is not necessary to provide a wearer-specific activation delay.

Referring still to FIG. 5A, when the lock-element 506 is in its low-temperature shape it is strongly curved, such that it applies a force against the top of the rail 112. Of course, alternative configurations may be envisaged in which the locking mechanism 500 presses one or more lock-elements 506 against one or both sides of the rail 112 instead of against the top of the rail 112, and/or in which the locking mechanism 500 presses lock-elements 506 against the rail 112 at more than one location. Regardless of the specific configuration of locking mechanism 500, the effect of the lock-element 506 pressing against the rail 112 is to create friction between the lock-element 506 and the surface of the rail 112, as well as between the rail 112 and the groove 114, when a force is applied to move the rail 112 along the displacement direction. This friction prevents the bottom plate 104 from sliding relative to the top plate 102 during normal handling of the device 100 when the device 100 is in its non-extended condition.

Referring now to FIG. 5B, after the device 100 is inserted into the wearer's mouth, body heat from the wearer is absorbed by the lock-element 506, which raises the temperature of the lock-element 506 above its transition temperature and causes the lock-element 506 to activate. Upon activating, the lock-element 506 substantially straightens such that the pressing force being applied to the rail 112 decreases, preferably to zero, and the lower plate 104 becomes unlocked and can slide freely along the displacement direction when the SMM spring 402 subsequently activates. FIG. 5C and FIG. 5D are enlarged views of the locking mechanism 500 showing more clearly the change of shape of the lock-element 506 between its “remembered” low-temperature shape in FIG. 5C and its “remembered” high-temperature shape in FIG. 5D. Preferably, the lock-element 506 is fabricated from a suitable two-way shape memory material, such that when the device 100 is removed from the wearers mouth and the lock-element 506 cools below its activation temperature, it returns to its “remembered” low-temperature shape and locks the device 100 in its non-extended condition.

### 6. Third Control Mechanism

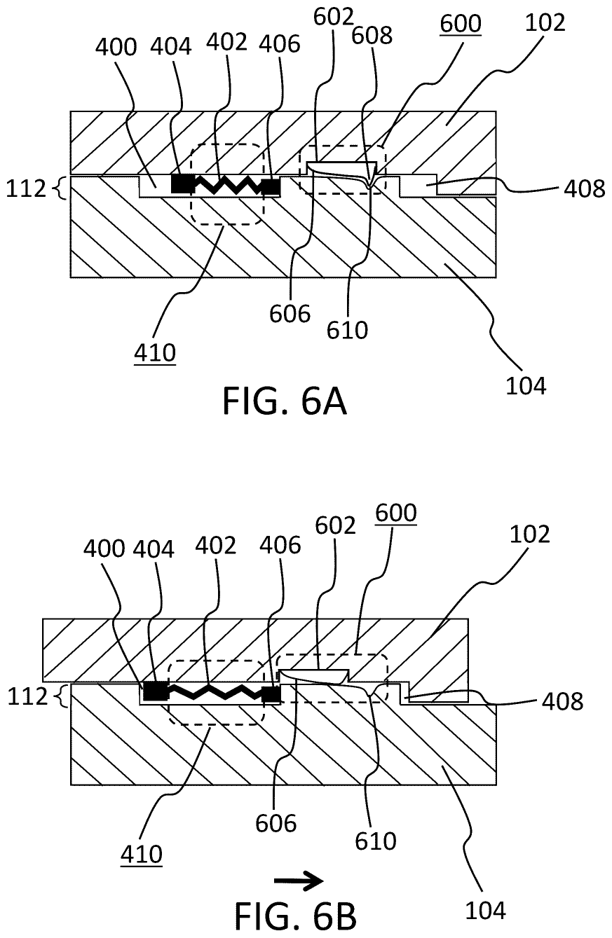

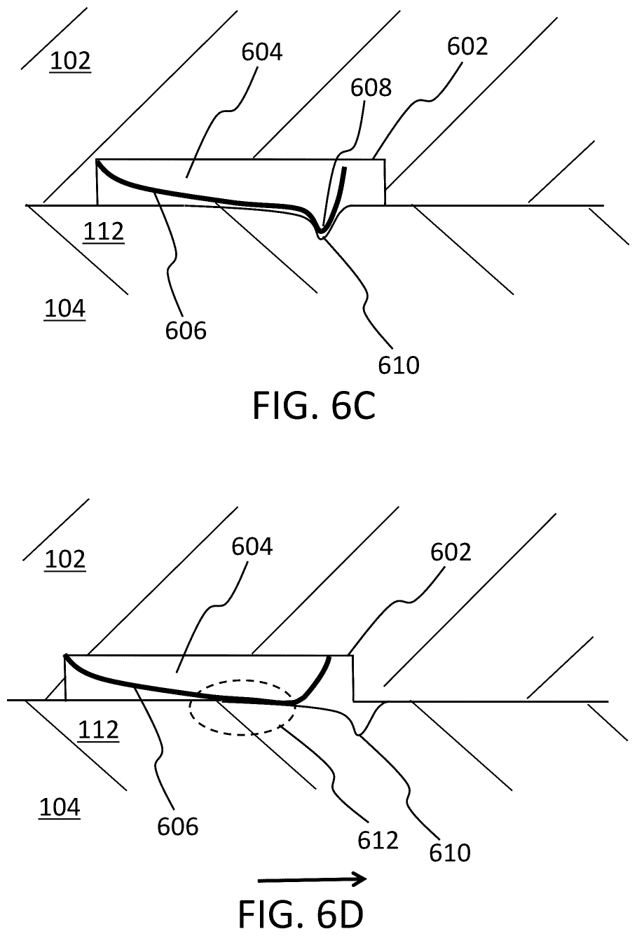

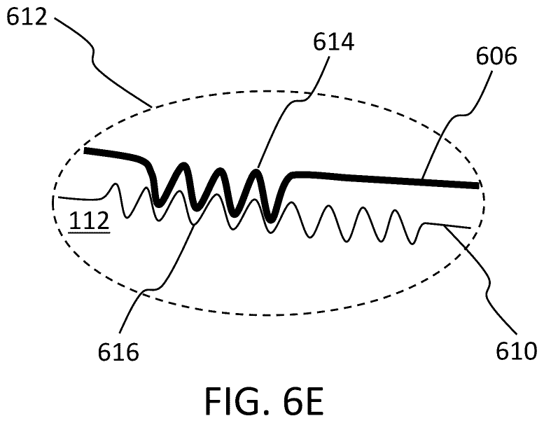

Referring now to FIG. 6A, shown is a cross-sectional view of device 100 taken along the line B-B in FIG. 1B, with a third control mechanism in accordance with an embodiment, and when the device 100 is in its non-extended condition. The elements of the third control mechanism include the same elements that have already been described with reference to FIG. 4A and FIG. 4B, and additionally include a locking mechanism shown generally at 600. During use, the locking mechanism 600 can be reset when the device 100 is returned to its non-extended condition after being worn by the wearer. When reset, i.e., in an engaged condition, the locking mechanism 600 locks the device 100 in its non-extended condition.

In this specific and non-limiting example, the locking mechanism 600 is disposed within a recess 602 that is formed in the groove 114 along the lower surface of the top plate 102. Now referring also to FIG. 6C and FIG. 6D, which show enlarged detail of the locking mechanism 600, the recess 602 provides a space 604 to accommodate a lock-element 606 formed from a SMM, such as for instance a curved piece of SMM having a tooth-feature 608 when in its “remembered” low-temperature shape. The tooth-feature 608 is received within a complementary shaped depression 610 formed along the top of rail 112. When the tooth-feature 608 is seated within depression 610, the device 100 is locked. Preferably, the locking mechanism 600 supplies a locking force that is greater than the locking force supplied by locking mechanism 500.

The lock-element 606 may be formed from the same material that is used to form SMM spring 402 or from another suitable shape memory material having an activation temperature close to average human body temperature. Optionally, the lock-element 606 is associated with a not illustrated heat-transfer control portion to provide an activation delay prior to activating, similar to the function of the already-described heat transfer portion 410. However, since the locking mechanism 600 is intended primarily to hold the device 100 in its non-extended condition only until after the device 100 has been inserted into the wearer's mouth, in most cases it is not necessary to provide an activation delay prior to activation of the lock-element 606, or at least it is not necessary to provide a wearer-specific activation delay.

Referring again to FIG. 6C and FIG. 6D, and now also to FIG. 6E, when the lock-element 606 is in its low-temperature shape the tooth-feature 608 seats within the depression 610 formed in the surface of rail 112 as discussed above. After being inserted into the wearer's mouth, and upon warming to its activation temperature, the shape of the lock-element 606 changes and the tooth-feature 608 “disappears” such that the shape of the lock-element 606 is more smoothly curved than when in its “remembered” low-temperature shape. This shape change unlocks the device. Optionally, as shown in FIG. 6D, the “remembered” high-temperature shape of the lock-element 606 results in contact between the lock-element 606 and the rail 112, such that a force is applied to the rail 112 that is sufficient to slow the displacement of the bottom plate 104 relative to the top plate 102, but not sufficient to prevent displacement entirely. This structure results in a less jarring change upon activation of the SMM spring 402 and thereby reduces the chance that the wearer will awaken when the device activates.

FIG. 6E shows an enlarged view of the features within the dashed line oval 612 of FIG. 6D. In particular, at least a portion of the lock-element 606 includes a fine, tooth-like structure 614 that is configured to slide against a correspondingly shaped fine, tooth-like structure 616 formed within at least a portion of the depression 610 along the surface of rail 112. The complementary fine, tooth-like structures 614 and 616 create a locking effect between the lock-element 606 and the rail 112, after the bottom plate 104 has been extended relative to the top plate 102, which prevents the bottom plate 104 from sliding backwards after it has advanced to its maximum extension position. Of course, when the device 100 is cooled after being removed from the wearer's mouth, the lock-element 606 returns to its “remembered” low temperature shape and the tooth-like structure 614 carried by the lock-element 606 either “disappears” or simply disengages from the tooth-like structure 616 formed on the at least a portion of the rail 112, which unlocks the device 100 allowing it to be reset to its non-extended condition.

### 7. Fourth Control Mechanism

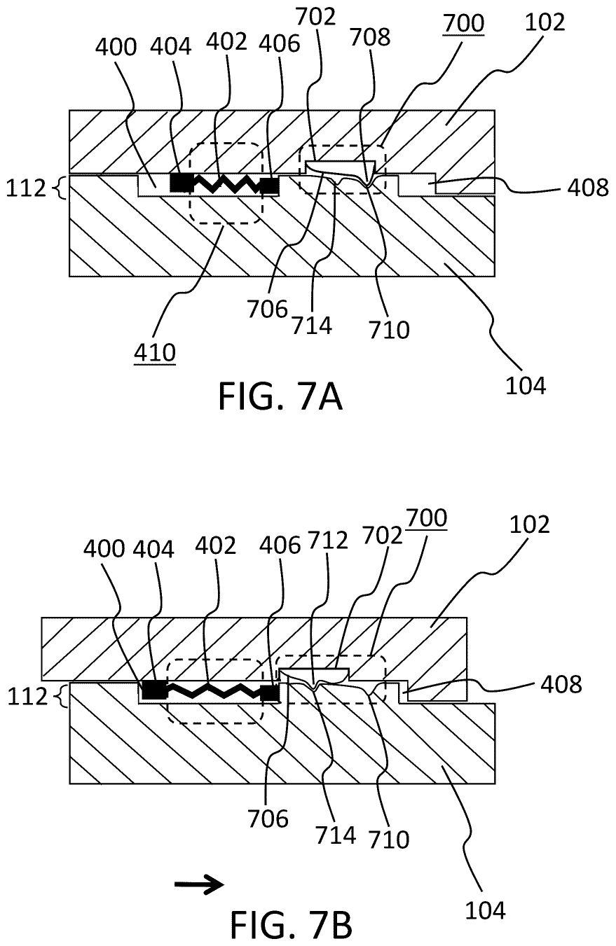

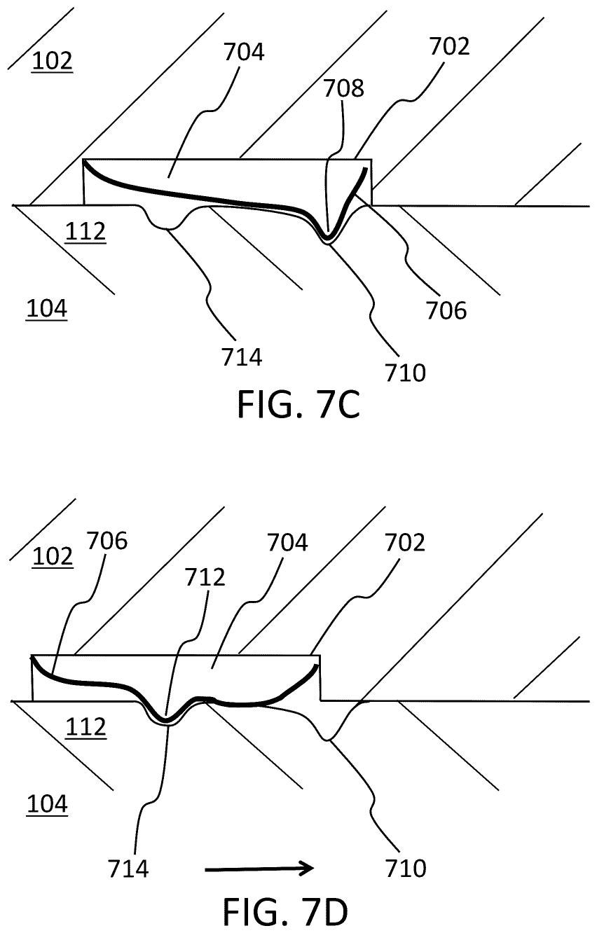

Referring now to FIG. 7A, shown is a cross-sectional view of device 100 taken along line B-B in FIG. 1B, with a fourth control mechanism in accordance with an embodiment, and when the device 100 is in its non-extended condition. The elements of the fourth control mechanism include the same elements that have already been described with reference to FIG. 4A and FIG. 4B, and additionally include a locking mechanism shown generally at 700. During use, the locking mechanism 700 can be reset when the device 100 is being returned to its non-extended condition after being worn by the wearer. When reset, i.e., in an engaged condition, the locking mechanism 700 locks the device 100 in its non-extended condition.

In this specific and non-limiting example, the locking mechanism 700 is disposed within a recess 702 that is formed in the groove 114 along the lower surface of the top plate 102. Now referring also to FIG. 7C and FIG. 7D, which show enlarged detail of the locking mechanism 700, the recess 702 provides a space 704 to accommodate a lock-element 706 formed from a SMM, such as for instance a curved piece of SMM having a tooth-feature 708 when in its “remembered” low-temperature shape. The tooth-feature 708 is received within a complementary shaped first depression 710 formed along the top of rail 112. When the tooth-feature 708 is seated within first depression 710, the device 100 is locked. The locking mechanism 700 is capable of supplying a locking force that is greater than the locking force supplied by locking mechanism 500.

After being inserted into the wearer's mouth, and upon warming to its activation temperature, the shape of the lock-element 706 changes and the tooth-feature 708 “disappears,” but concomitantly a second tooth feature 712 “appears.” This shape change unlocks the device 100 and allows the bottom plate 104 to slide relative to the top plate 102. When the device is in its full extended condition, the second tooth-feature 712 engages a second depression 714 formed along the top of rail 112. When the second tooth-feature 712 is seated within second depression 714, the device 100 is locked. Of course, the “remembered” high-temperature shape of the lock-element 706 should be programmed such that the second tooth-feature 712 is located at a position that allows it to seat within the second depression 714 when the device 100 is extended to the maximum extension for that particular wearer. Since the lock-element 706 is somewhat flexible, precise location of the second tooth-feature 712 is not absolutely critical.

Optionally, as shown in FIG. 7D, the “remembered” high-temperature shape of the lock-element 706 results in contact between the lock-element 706 and the rail 112, such that a force is applied to the rail 112 that is sufficient to slow the displacement of the bottom plate 104 relative to the top plate 102, but not to prevent the displacement entirely. This structure results in a less jarring change upon activation of the SMM spring 402 and thereby reduces the chance that the wearer will awaken when the device activates.

The lock-element 706 may be formed from the same material that is used to form SMM spring 402 or from another suitable shape memory material having an activation temperature close to average human body temperature. Optionally, the lock-element 706 is associated with a not illustrated heat-transfer control portion to provide an activation delay prior to activating, similar to the function of the already-described heat transfer portion 410. However, since the locking mechanism 700 is intended primarily to hold the device 100 in its non-extended condition only until after the device 100 has been inserted into the wearer's mouth, in most cases it is not necessary to provide an activation delay prior to activation of the lock-element 706, or at least it is not necessary to provide a wearer-specific activation delay.

### 8. Fifth Control Mechanism

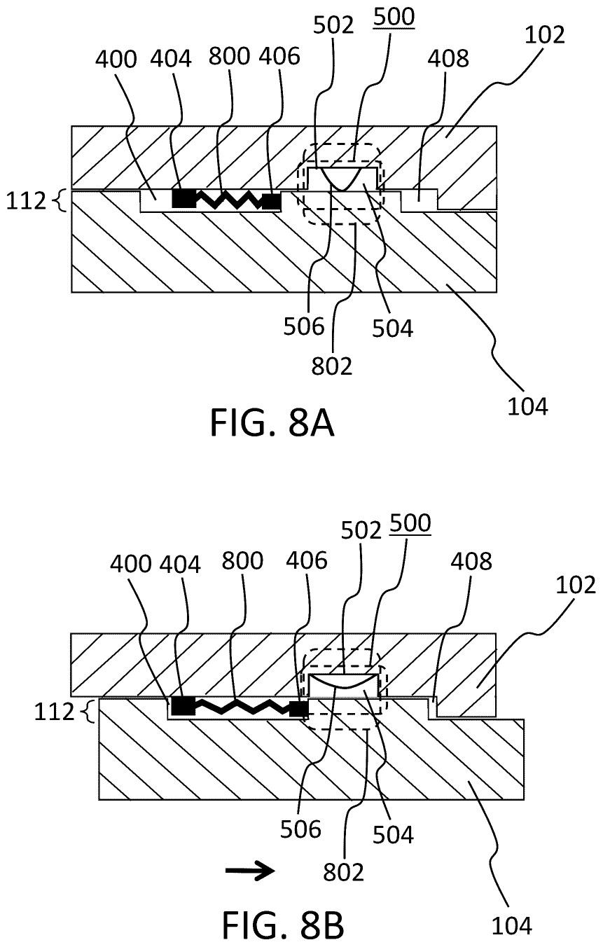

Referring now to FIG. 8A, shown is a cross-sectional view of device 100 taken along line B-B in FIG. 1B, with a fifth control mechanism in accordance with an embodiment, and when the device 100 is in its non-extended condition. FIG. 8B shows the device in its extended condition. The embodiment shown in FIG. 8A is substantially similar to the embodiment shown in FIG. 5A, however the SMM spring 402 of FIG. 5A has been replaced with a helical compression spring 800 that is not fabricated using a shape memory material. As such, the spring 800 provides a constant force that is trying to slide the lower plate 104 relative to the top plate 102, to move the bottom plate 102 to its extended position. The locking mechanism 500 is configured to provide a sufficiently large force to counteract the force of the spring 800 when the lock-element 506 is in its low temperature shape. As shown in FIG. 8A, the heat transfer control portion 410 used to control activation of SMM spring 402 in the embodiment of FIG. 5A is omitted, and instead a heat transfer portion 802 is provided for controlling the activation of the locking mechanism 500.

Heat transfer portion 802 may be configured similarly to heat transfer portion 410, i.e., by selecting appropriate material compositions, material thicknesses, using various fluids and/or solids etc. to control the rate at which the wearer's body heat is transferred to and absorbed by the lock-element 506. In this way, the device 100 may be configured to impose a desired activation delay after being inserted into the wearer's mouth before the lock-element 506 is warmed to its activation temperature and changes shape, thereby releasing rail 112 and allowing spring 800 to extend and displace the bottom plate 104 to its fully extended position.

Preferably, the lock-element 506 is fabricated from a suitable two-way shape memory material, such that when the device 100 is removed from the wearers mouth and the lock-element 506 cools below its activation temperature, it returns to its “remembered” low-temperature shape and locks the device 100 in its non-extended condition.

### 9. Sixth Control Mechanism

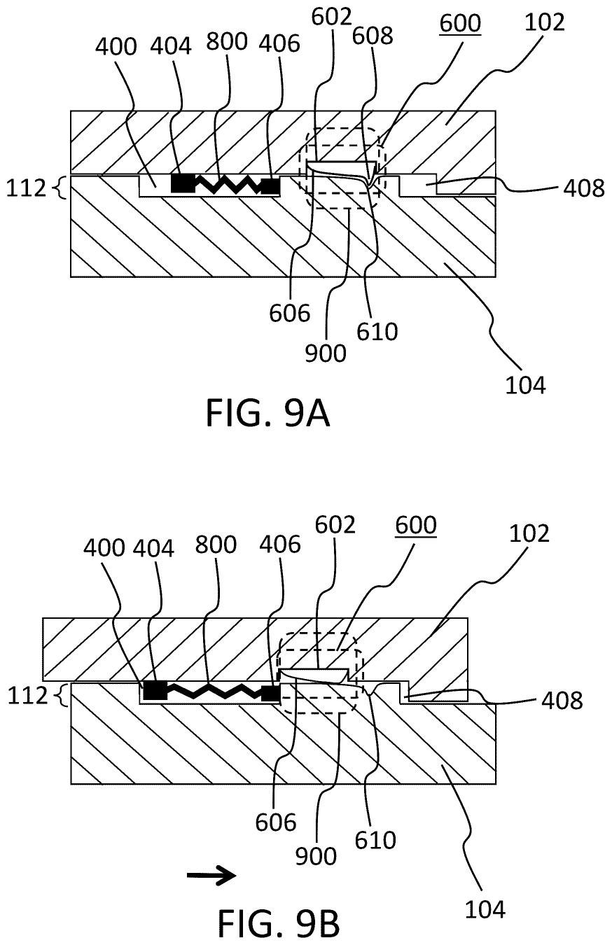

Referring now to FIG. 9A, shown is a cross-sectional view of device 100 taken along line B-B in FIG. 1B, with a sixth control mechanism in accordance with an embodiment, and when the device 100 is in its non-extended condition. FIG. 9B shows the device in its extended condition. The embodiment shown in FIG. 9A is substantially similar to the embodiment shown in FIG. 6A, however the SMM spring 402 of FIG. 6A has been replaced with a helical compression spring 800 that is not fabricated using a shape memory material. As such, the spring 800 provides a constant force that is trying to slide the lower plate 104 relative to the top plate 102, to move the bottom plate 102 to its extended position. The locking mechanism 600 is configured to provide a sufficiently large force to counteract the force of the spring 800 when the lock-element 606 is in its low temperature shape. As shown in FIG. 9A, the heat transfer control portion 410 used to control activation of SMM spring 402 in the embodiment of FIG. 6A is omitted, and instead a heat transfer portion 900 is provided for controlling the activation of the locking mechanism 600.

Heat transfer portion 900 may be configured similarly to heat transfer portion 410, i.e., by selecting appropriate material compositions, material thicknesses, using various fluids and/or solids etc. to control the rate at which the wearer's body heat is transferred to and absorbed by the lock-element 606. In this way, the device 100 may be configured to impose a desired activation delay after being inserted into the wearer's mouth before the lock-element 606 is warmed to its activation temperature and changes shape, thereby releasing rail 112 and allowing spring 800 to extend and displace the bottom plate 104 to its fully extended position.

Optionally, as discussed above with reference to FIG. 6D, the “remembered” high-temperature shape of the lock-element 606 may be configured to result in contact between the lock-element 606 and the rail 112, such that a force is applied to the rail 112 that is sufficient to slow the displacement of the bottom plate 104 relative to the top plate 102, but not sufficient to prevent displacement entirely. This structure results in a less jarring change upon activation of the lock-element 606 and release of the stored potential energy in spring 800, and thereby reduces the chance that the wearer will awaken when the device activates.

Preferably, the lock-element 606 is fabricated from a suitable two-way shape memory material, such that when the device 100 is removed from the wearers mouth and the lock-element 606 cools below its activation temperature, it returns to its “remembered” low-temperature shape and locks the device 100 in its non-extended condition.

### 10. Seventh Control Mechanism

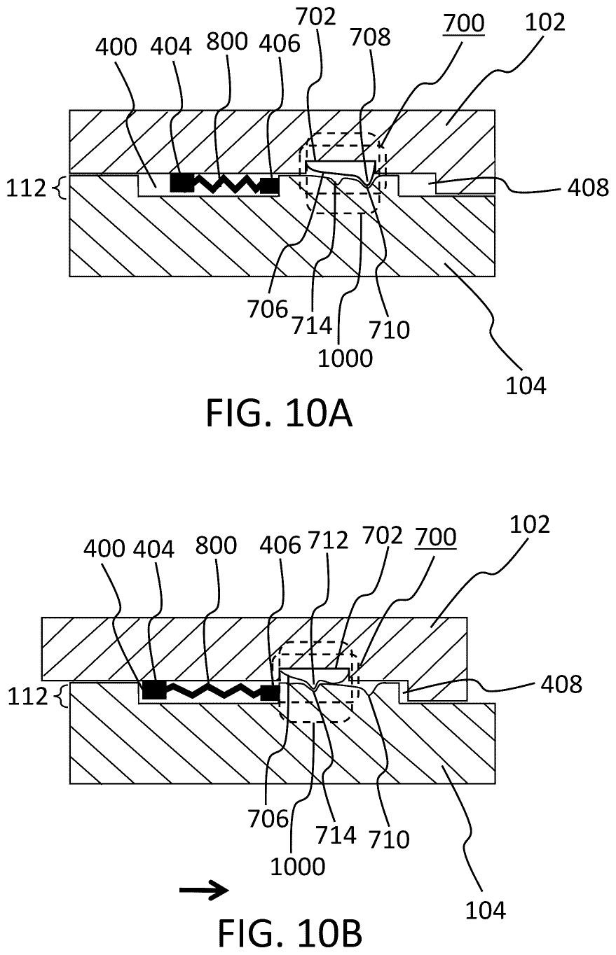

Referring now to FIG. 10A, shown is a cross-sectional view of device 100 taken along line B-B in FIG. 1B, with a seventh control mechanism in accordance with an embodiment, and when the device 100 is in its non-extended condition. FIG. 10B shows the device in its extended condition. The embodiment shown in FIG. 10A is substantially similar to the embodiment shown in FIG. 7A, however the SMM spring 402 of FIG. 7A has been replaced with a helical compression spring 800 that is not fabricated using a shape memory material. As such, the spring provides a constant force that is trying to slide the lower plate 104 relative to the top plate 102, to move the bottom plate 102 to its extended position. The locking mechanism 700 is configured to provide a sufficiently large force to counteract the force of the spring 800 when the lock-element 706 is in its low temperature shape. As shown in FIG. 10A, the heat transfer control portion 410 used to control activation of SMM spring 402 in the embodiment of FIG. 7A is omitted, and instead a heat transfer portion 1000 is provided for controlling the activation of the locking mechanism 700.

Heat transfer portion 1000 may be configured similarly to heat transfer portion 410, i.e., by selecting appropriate material compositions, material thicknesses, using various fluids and/or solids etc. to control the rate at which the wearer's body heat is transferred to and absorbed by the lock-element 706. In this way, the device 100 may be configured to require a desired activation delay after being inserted into the wearer's mouth before the lock-element is warmed to its activation temperature and changes shape, thereby releasing rail 112 and allowing spring 800 to extend and displace the bottom plate 104 to its fully extended position.

Optionally, the “remembered” high-temperature shape of the lock-element 706 results in contact between the lock-element 706 and the rail 112, such that a force is applied to the rail 112 that is sufficient to slow the displacement of the bottom plate 104 relative to the top plate 102, but not to prevent the displacement entirely. This structure results in a less jarring change upon activation of the lock-element 706 and thereby reduces the chance that the wearer will awaken when the device activates.

Preferably, the lock-element 706 is fabricated from a suitable two-way shape memory material, such that when the device 100 is removed from the wearers mouth and the lock-element 706 cools below its activation temperature, it returns to its “remembered” low-temperature shape and locks the device 100 in its non-extended condition.

### 11. Heat Transfer Control Portion

As discussed in the preceding sections, the heat transfer control portion 410 used to control the rate of heat transfer from the wearer to the SMM spring 402, and equivalently the heat transfer control portion 802, 900 or 1000 used to control the rate of heat transfer from the wearer to the lock-element 506, 606 or 706, may be provided in the form of a flexible, liquid-tight polymer such as polyvinylidene chloride, i.e., a plastic film/membrane. FIGS. 11A-11H and 12A-12D explicitly depict the heat transfer control portion 410, but the same principles may also be applied to the heat transfer control portion 802, 900 or 1000.

FIGS. 11A, 11C, 11E, and 11G are simplified diagrams showing a heat transfer control portion 410 comprising a flexible film/membrane 1200 configured to contain a shape memory material (SMM) spring 402 and a heat transfer control gas 1202 in FIG. 11A, a heat transfer control liquid 1204 in FIG. 11C, a heat transfer control gel 1206 in FIG. 11E, and a solid insulator material 1208 such as for instance Styrofoam® beads in FIG. 12G. The heat transfer control portion 410 in each case is shown in a condition prior to warming the SMM spring 402 to its activation temperature.

FIGS. 11B, 11D, 11F and 11H are simplified diagrams showing the heat transfer control portion 410 of FIGS. 11A, 11C, 11E, and 11G, respectively, in a condition after warming the SMM spring 402 to its activation temperature. In this specific example, the SMM spring 402 elongates upon activation and the flexible film/membrane 1200 changes shape.

In each case, the flexible film/membrane 1200 is coupled to the attachment points 404 and 406 in a fluid-tight fashion. Various methods for forming suitable fluid-tight connections are known in the art.

An alternative configuration of the heat transfer control portion 410 is shown in FIGS. 12A-12D, in which the flexible film/membrane 1200 forms a tube-shaped enclosure with a central passage 2010 within which the SMM spring 402 is received. The enclosure containing a heat transfer control gas 1202 in FIG. 12A, a heat transfer control liquid 1204 in FIG. 12B, a heat transfer control gel 1206 in FIG. 12C, and a solid insulator material 1208 such as for instance Styrofoam® beads in FIG. 12D.

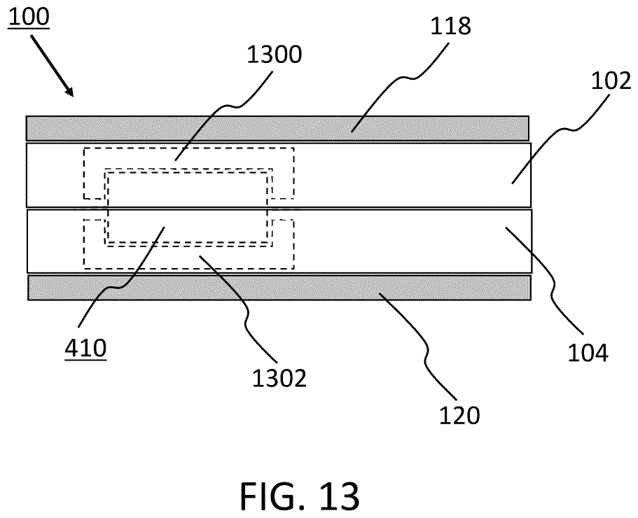

FIG. 13 is a simplified diagram showing yet another alternative configuration of device 100. As shown in FIG. 13, the heat transfer control portion described previously may be at least partially surrounded or shielded by additional thermal material 1300 and 1302 disposed between the SMA spring 402 (not shown in FIG. 13) and the exterior surfaces of the device 100 which come into contact with portions of the wearer's mouth. The thermal material 1300 and/or 1302 have a material composition and/or material thickness etc. that is selected to provide a desired activation delay. The additional thermal material 1300 and 1302 may be a solid, liquid or gas, or combinations thereof. Optionally, the additional thermal material 1300 and/or 1302 is user changeable, for allowing the wearer to make fine adjustments to the delay time without the need to return the device to be professionally adjusted.

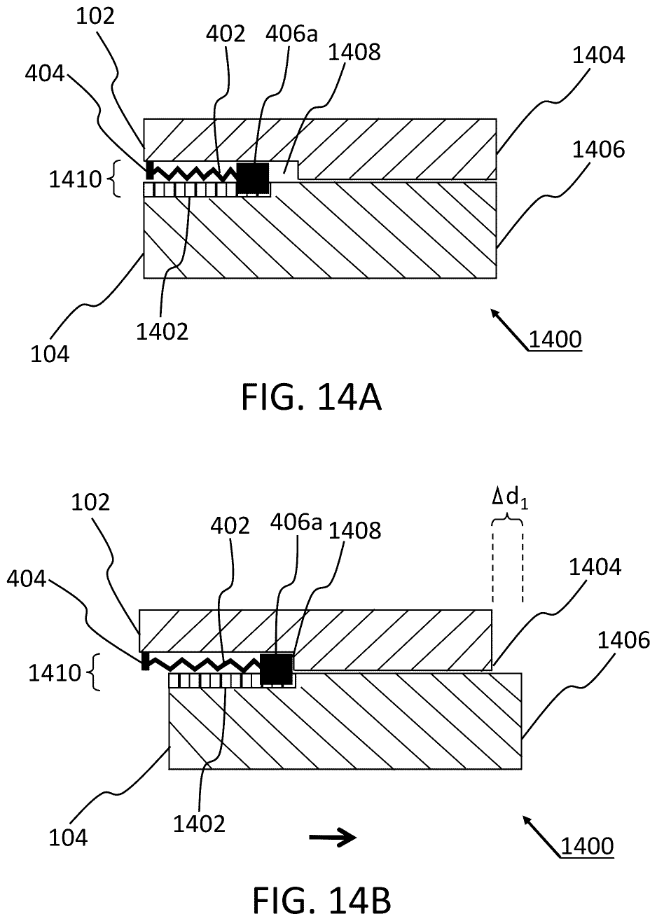

Referring now to FIGS. 14A and 14B, shown are enlarged cross-sectional views of a configurable, time-delayed oral mandible displacement device 1400 according to an embodiment. The device 1400 includes an alternative coupling and actuation portion, shown generally at 1410, which allows a user to set an initial position of the bottom plate 104 relative to the top plate 102, within a range of selectable positions. The views that are shown in FIGS. 14A and 14B are taken through a portion of the device 1400 that is similar to the portion indicated by line B-B in FIG. 1B.

In the various embodiments that have been described in the preceding paragraphs, an SMM spring 402 is attached to the top plate 102, also referred to equivalently as the top member 102, via attachment point 404, and is attached to the bottom plate 104, also referred to equivalently as the bottom member 104, via an attachment point 406. In the previously described embodiments, the position of the attachment point 404 is fixed relative to the top plate 102 and the position of the attachment point 406 is fixed relative to the bottom plate 104.

In the instant embodiment that is shown in FIGS. 14A and 14B, the location of an attachment point 406a, also referred to as anchor 406a, is adjustable relative to the bottom plate 104. In this illustrative example, for improved clarity, the attachment point 404 is fixed relative to the top plate 102. Of course, alternative configurations may be envisaged in which the location of the attachment point 404 is adjustable relative to the top plate 102. As will be apparent, the ability to adjust the position of the attachment point 406a allows the initial relative position of the top and bottom plates to be adjusted. Since different users have differently-shaped mouths, and may be bothered to different degrees by the fit of the device 1400, the ability to be able to change the initial position of the bottom plate 104 relative to the top plate 102 can lead to improved comfort and better outcomes for the wearer.

In the embodiment that is shown in FIGS. 14A and 14B, the top plate 102 and the bottom plate 104 are both substantially U-shaped to match the dentition of the wearer of the device, also referred to equivalently as the user or as the human user. The top plate 102 and the bottom plate 104 may be fabricated using any material that is generally suitable for use in oral appliance applications, such as for instance medical grade PVC or Polyethylene, PEEK, Polycarbonate, Polypropylene and Polyurethane, etc.

Prior to use by the user, for instance during a fitting appointment, the location of the anchor 406a is set to provide a desired initial position of the bottom plate 104 relative to the top plate 102. In this exemplary embodiment, the anchor 406a is seated within and retained by a complementary mating structure 1402 formed within the bottom plate 104. The complementary mating structure 1402 supports a plurality of discrete locations for receiving the anchor 406a.

FIG. 14A shows the anchor 406a received at a location within the mating structure 1402 that results in an initial position in which the front ends 1404 and 1406 of the top and bottom plates 102 and 104, respectively, are substantially aligned. FIG. 14B shows the front end 1406 of the bottom plate 104 displaced by a distance Δd₁ to its final position, after activation of the device 1400 by the body heat of the user. The force required to displace the bottom plate 104 relative to the top plate 102 may be provided at least in part by the spring 402. Preferably, spring 402 is formed from a SMM alloy, which changes shape and lengthens upon heating to its activation temperature, thereby providing force to slide the bottom plate 104 frontward, relative to the top plate 102. Optionally, an additional (not illustrated) spring may provide at least a part of the force that is required to displace the bottom plate 104 relative to the top plate 102. Optionally, the additional spring may be fabricated from a SMM alloy or from a non-SMM alloy.

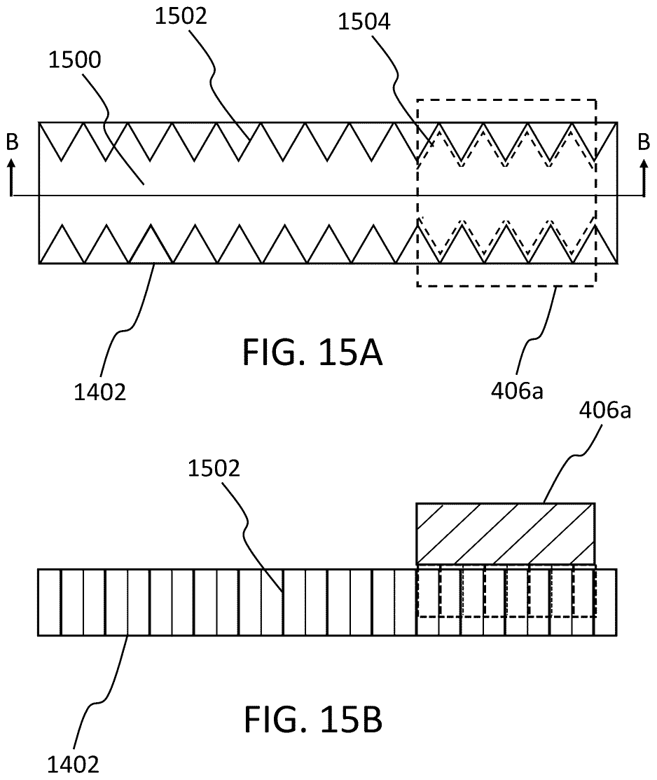

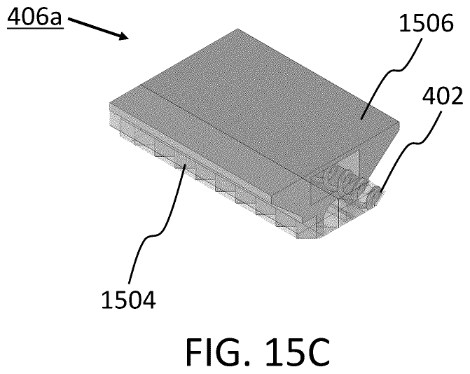

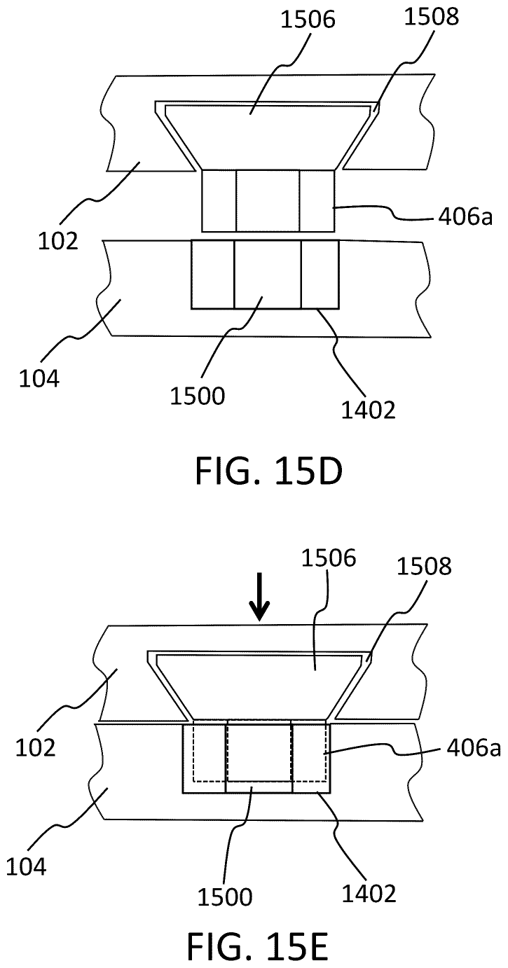

FIG. 15A is a partial top view showing the structure of the complementary mating structure 1402. Hidden portions of the anchor 406a are shown using broken lines for improved clarity. In this exemplary embodiment, the complementary mating structure 1402 is a groove or recess 1500 having opposed, toothed side-surfaces 1502, which receives and retains the teeth 1504 that are formed along the side surfaces of the anchor 406a, in an interference fit manner.

FIG. 15B is a cross-sectional view taken along the line B-B in FIG. 15A, showing the anchor 406a retained by the complementary mating structure 1402.

FIG. 15C is a simplified isometric view showing an exemplary anchor 406a, including the teeth 1504 that are formed along the side surfaces of a lower portion thereof. In this embodiment the anchor 406a includes an enlarged upper portion 1506 having the shape of an isosceles trapezoid. As is shown more clearly in FIGS. 15D and 15E, the enlarged upper portion 1506 of the anchor 406a is retained within and slides along a correspondingly shaped recess 1508 that is formed in the lower side of the top plate 102. FIGS. 15D and 15E also show the manner in which the anchor 406a is press-fit into the groove 1500 of complementary mating structure 1402. When the device 1400 is in the assembled condition that is shown in FIG. 15E, the top and bottom plates 102 and 104 are held together by the anchor 406a but are slidable relative to one another in a direction perpendicular to the plane of the page.

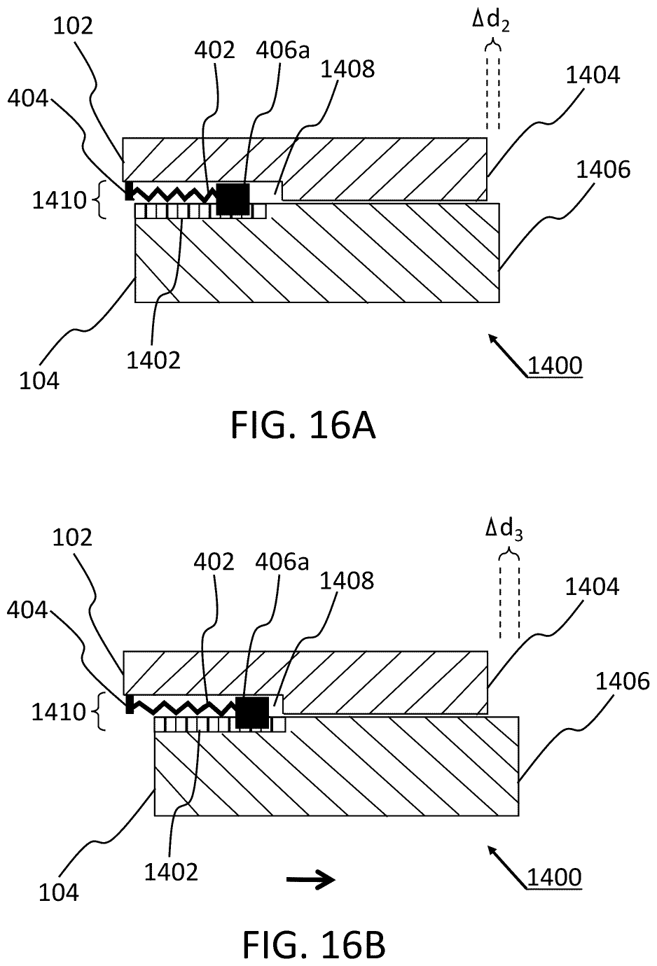

FIGS. 16A and 16B show the same device that is shown in FIGS. 14A and 14B, but with the anchor 406a received at a different location within the complementary mating structure 1402, which results in an initial position in which the front ends 1404 and 1406 of the top and bottom plates 102 and 104, respectively, are not substantially aligned. Referring specifically to FIG. 16A, the front end 1406 of the bottom plate 104 protrudes beyond the front end 1404 of the top plate 102 by an amount Δd₂. FIG. 16B shows the front end 1406 of the bottom plate 104 displaced by a further distance Δd₃, which is smaller than Δd₁ in FIG. 14B, to its final position after activation of the device 1400 by the body heat of the user. Although the final position of the bottom plate 104 relative to the top plate 102 is the same in FIG. 14B and in FIG. 16B (i.e., Δd₁≈Δd₂+Δd₃), the displacement of the bottom plate 104 during use is smaller due to the differences in the initial position of the bottom plate 104 relative to the top plate 102.

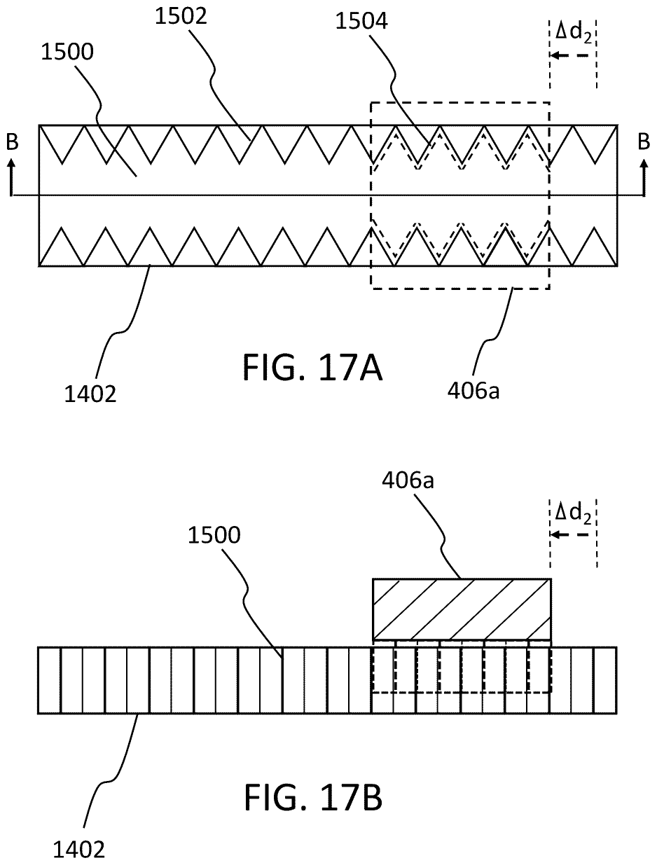

FIGS. 17A and 17B are similar to FIGS. 15A and 15B, but with the anchor 406a inserted into the complementary structure 1402 at a location corresponding to the configuration that is shown in FIGS. 16A and 16B. In FIGS. 17A and 17B, the anchor 406a has been moved a distance Δd₂, which causes the bottom member 104 to shift by approximately a corresponding amount such that the front end 1404 thereof protrudes beyond the front end 1402 of the top member 102 by Δd₂. As will be apparent, increasing the magnitude of the displacement Δd₂ causes the front end 1404 of the bottom member 104 to protrude further beyond the front end 1402 of the top member 102 when in the initial position. In this fashion, the device 1400 may be customized to provide different initial positions for different users, thereby optimizing comfort and improving outcomes, etc. After being inserted into the user's mouth, the device 1400 remains in the initial condition until sufficient body heat has been transferred to warm the SMM material of the spring 402 to its activation temperature, cause a shape change that provides at least part of the force required to fully extend the bottom member 104 relative to the top member 102.

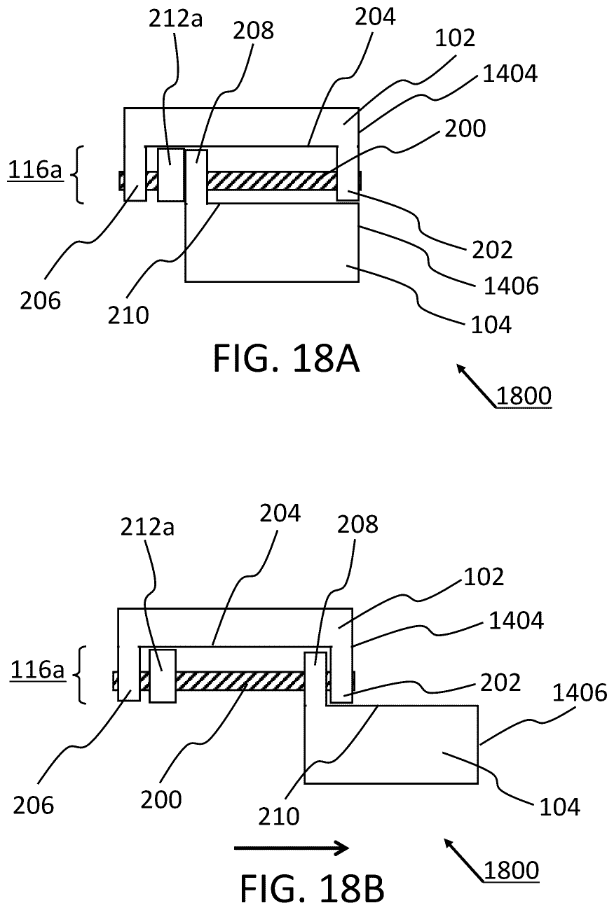

FIGS. 18A and 18B, which are similar to FIGS. 2A and 2B, show cross-sectional views illustrating an alternative screw-adjustment mechanism 116a. FIG. 18A shows a device 1800 in its non-extended condition and FIG. 18B shows the device 1800 in its fully extended condition. Similar to the screw-adjustment mechanism 116, the screw-adjustment mechanism 116a also includes a threaded screw 200 that is retained within respective openings, at least some of which are also threaded, formed through a first post 202 extending from a lower surface 204 of the top plate 102, a second post 206 extending from the lower surface 204 of the top plate 102, and a third post 208 extending from the upper surface 210 of the bottom plate 104. An adjustment block 212a, also referred to equivalently as a stop member 212a, moves along the length of the threaded screw 200 when the threaded screw 200 is turned. Unlike the configuration that is shown in FIGS. 2A and 2B, in which case the stop member 212 is disposed on a side of the third post 208 for controllably varying a maximum final extension of the bottom plate 104, FIGS. 18A and 18B show the stop member 212a on the opposite side of the third post 208 for controllably varying an initial displacement of the bottom plate 104 relative to the top plate 102.

In FIGS. 18A and 18B, the stop member 212a is disposed at a location along the length of the threaded screw 200 such that the front ends 1404 and 1406 of the top and bottom plates 102 and 104, respectively, are substantially aligned. FIG. 18A shows the initial condition of the device 1800 prior to being placed into the user's mouth. When the device 1800 is in the initial condition, the stop member 212a and the third post 208 extending from the upper surface 210 of the bottom plate 104 come into contact one with the other and thereby define the initial displacement of the bottom plate 104 relative to the top plate 102, in this case “zero” displacement. FIG. 18B shows the device 1800 in the fully extended condition, after being warmed by body heat transferred from the user to the device 1800, which raises the temperature of (not illustrated) springs or other force providing elements that are fabricated from a SMM, which undergo a shape change above a known activation temperature.

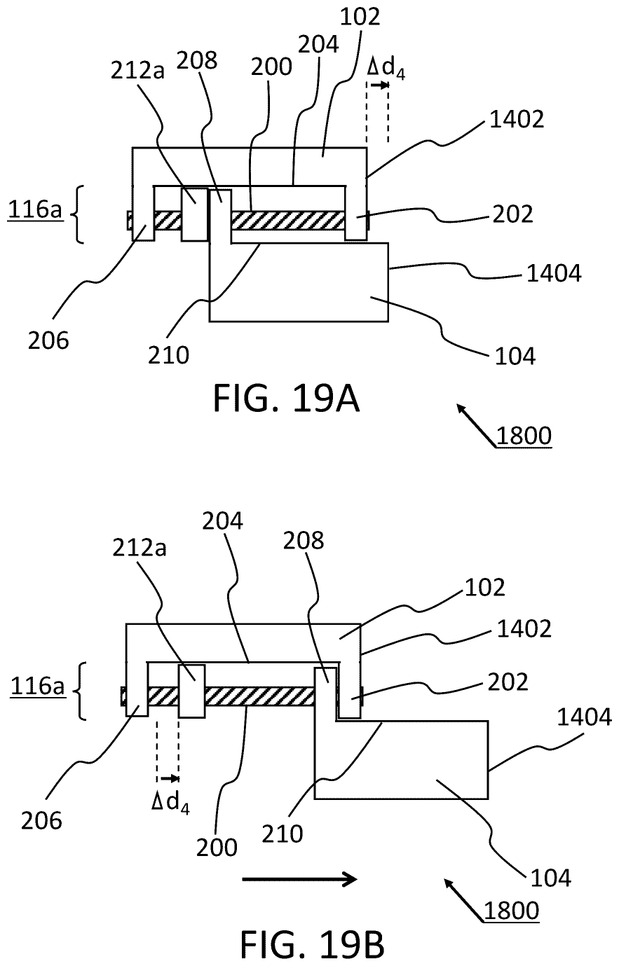

FIGS. 19A and 19B show the same screw-adjustment mechanism 116a that is shown in FIGS. 18A and 18B, but in FIGS. 19A and 19B the stop member 212a is disposed at a location along the length of the threaded screw 200 such that the front end 1406 of the bottom plate protrudes past the front end 1404 of the top plate 102 when the device is in its initial condition. More specifically, the threaded screw 200 has been turned by a sufficient amount to advance the stop member 212a along the length of the threaded screw by the distance Δd₄ as shown in FIG. 19B. This adjustment causes the front end 1406 of the bottom plate to protrude past the front end 1404 of the top plate 102 by a similar distance Δd₄ as shown in FIG. 19A. As will be apparent, increasing the magnitude of the displacement Δd₄ causes the front end 1406 of the bottom member 104 to protrude further beyond the front end 1404 of the top member 102 when in the initial position. In this fashion, the device 100 may be customized to provide different initial positions for different users, thereby optimizing comfort and improving outcomes, etc. After being inserted into the user's mouth, the device 100 remains in the initial condition until sufficient body heat has been transferred to warm the SMM material of the spring 402 to its activation temperature, cause a shape change that provides at least part of the force required to fully extend the bottom member 104 relative to the top member 102.

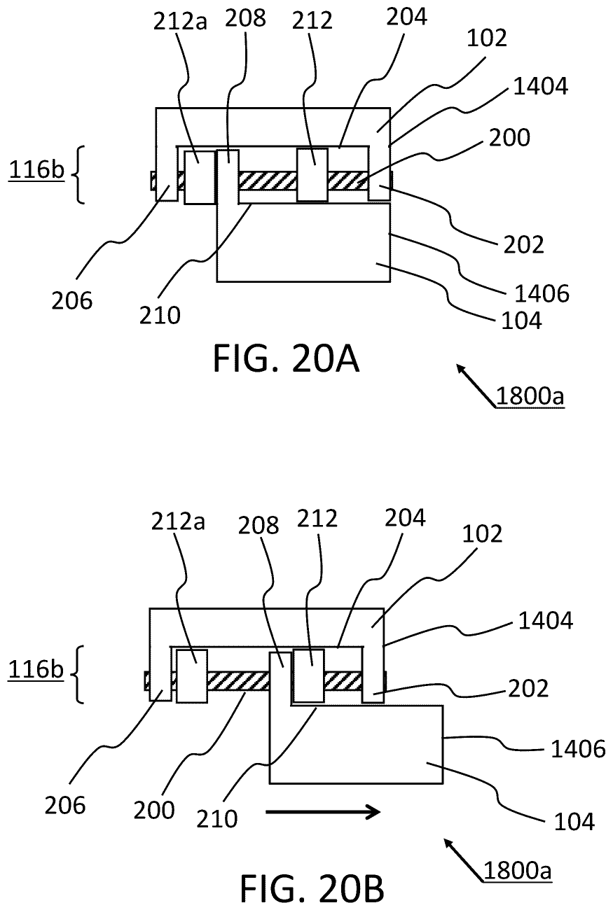

FIGS. 20A and 20B, which are similar to FIGS. 2A, 2B and 18A-19B, show cross-sectional views illustrating another alternative screw-adjustment mechanism 116b. FIG. 20A shows a device 1800a in its non-extended condition and FIG. 20B shows the device 1800a in its fully extended condition. The alternative screw-adjustment mechanism 116b also includes a threaded screw 200 that is retained within respective openings, at least some of which are also threaded, formed through a first post 202 extending from a lower surface 204 of the top plate 102, a second post 206 extending from the lower surface 204 of the top plate 102, and a third post 208 extending from the upper surface 210 of the bottom plate 104. The alternative screw-adjustment mechanism 116b includes a first adjustment block 212, also referred to equivalently as a first stop member 212, which is mounted on the threaded screw 200 and which is disposed on the front side of the third post 208. The alternative screw-adjustment mechanism 116b further includes a second adjustment block 212a, also referred to equivalently as a second stop member 212a, which is mounted on the same threaded screw 200, and which is disposed on the back side of the third post 208, opposite the front side.

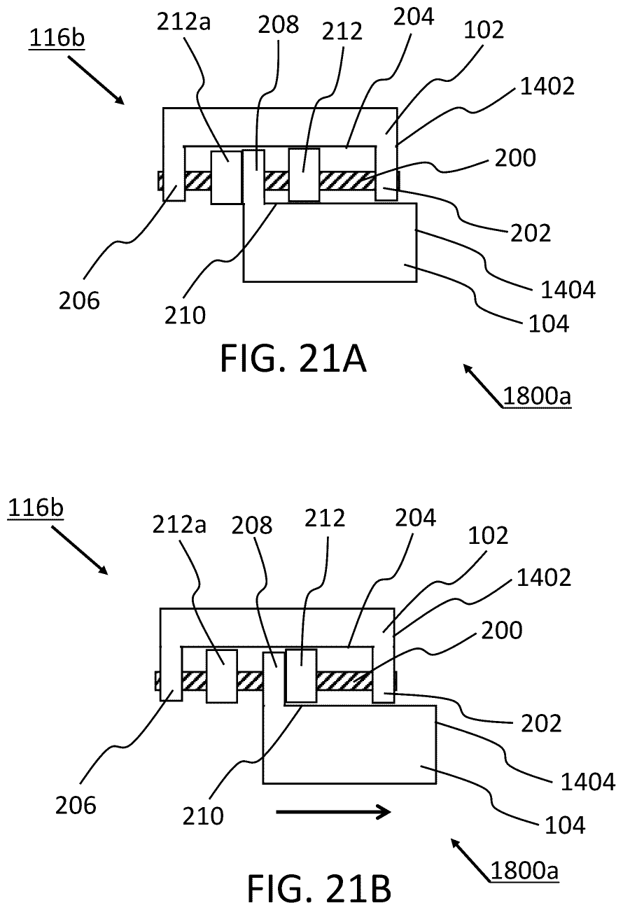

Changing the position of the first adjustment block 212 along the length of the threaded screw 200 changes the final position of the lower plate 104 relative to the top plate 102, whilst changing the position of the second adjustment block 212a along the length of the threaded screw 200 changes the initial position of the lower plate 104 relative to the top plate 102. As will be apparent, the device 1800a is fully adjustable, in terms of the initial and final positions of the lower plate 104 relative to the top plate 102, using a single, threaded adjustment screw 200. For instance, FIGS. 21A and 21B show the device 1800a after changing the position of the first adjustment block 212 and changing the position of the second adjustment block 212a, compared to the positions that are shown in FIGS. 20A and 20B. In the initial position shown in FIG. 20A, the front ends 1404 and 1406 of the top and bottom plates 102 and 104, respectively, are substantially aligned whereas in the initial position shown in FIG. 21A the front end 1406 of the bottom plate 104 protrudes past the front end 1404 of the top plate 102. In the final position shown in FIG. 20B the front end 1406 of the bottom plate 104 extends beyond the front end 1404 of the top plate 102 by a larger distance than in the final position that is shown in FIG. 21B.

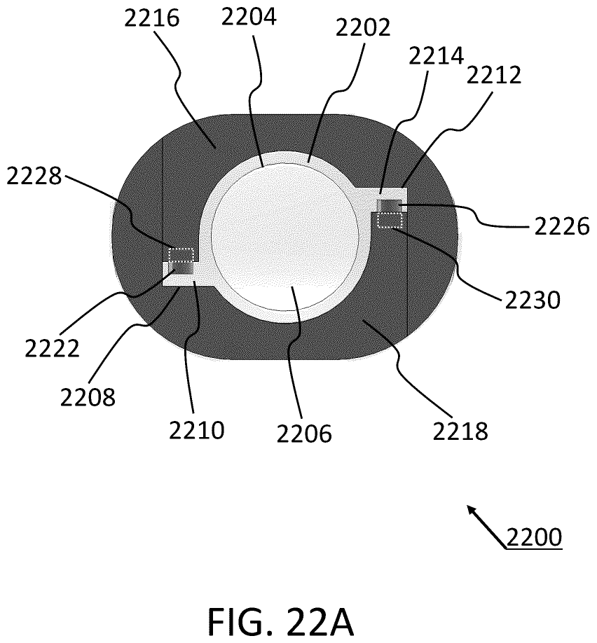

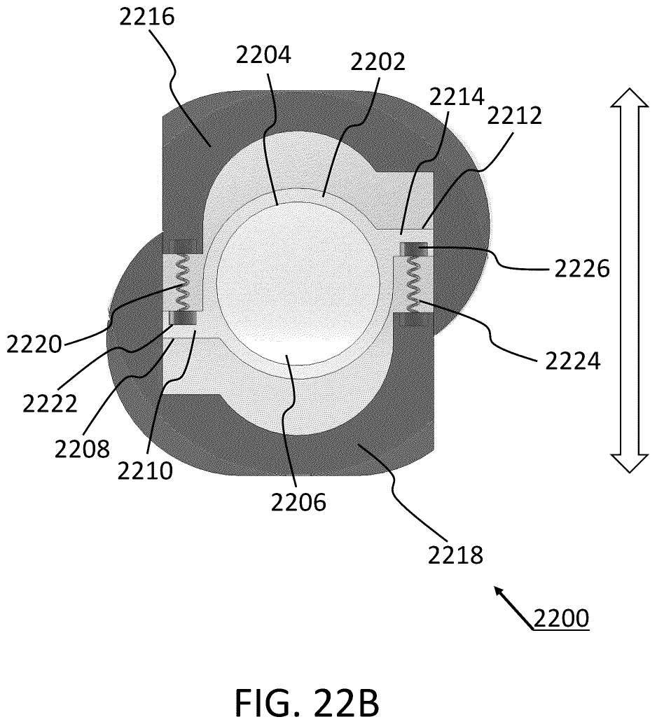

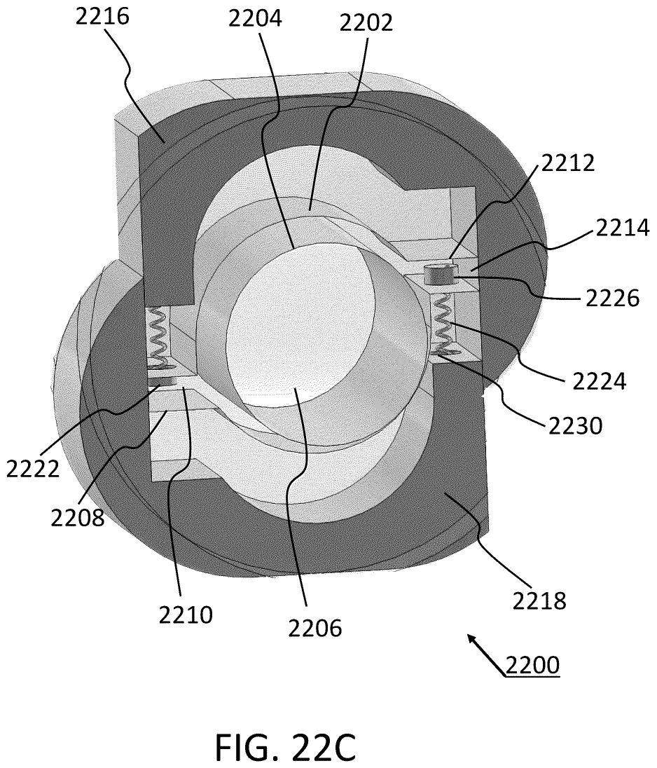

Adjusting both the initial and final positions of the top and bottom plates 102 and 104 using a single threaded adjustment screw 200 is made possible in the embodiment that is shown in FIGS. 20A and 20B and in FIGS. 21A and 21B by the use of a two-way SMM having two “remembered” shapes and 2 distinct activation temperatures (e.g. room temperature and close to body temperature), as will be discussed below in more detail with reference to FIGS. 22A through 22C.

FIGS. 22A, 22B and 22C show an expandable adjustment block 2200 for use with the embodiments that are shown in FIGS. 20A-21B. One feature of the expandable adjustment block is that it contains elements, such as for instance springs, that are fabricated from a two-way SMM. Heating and cooling the two-way SMM allows the user to toggle the expandable adjustment block 2200 between an adjustable condition and a non-adjustable condition.

The expandable adjustment block 2200 includes a base element 2202 with a generally circular central portion 2204 having a through-hole 2206 with an internal thread (not shown). The through-hole 2206 and internal thread are configured to engage the external thread of the threaded screw 200. A first pair of plates 2208 with a space 2210 formed therebetween extends from one side of the generally circular central portion 2204. Similarly, a second pair of plates 2212 with a space 2214 defined therebetween extends from the opposite side of the generally circular central portion 2204. As is shown in FIG. 22A, the first pair of plates 2208 and the second pair of plates 2212 are not diametrically opposite one another, and they do not extend perpendicularly from the generally circular central portion 2204.

A first outer element 2216 and a second outer element 2218 are disposed symmetrically about the generally circular central portion 2204. The first outer element 2216 is coupled to the generally circular central portion 2204 via SSM spring 2220, which is shown in FIGS. 22B and 22C but not in FIG. 22A, and which is anchored to the generally circular central portion 2204 via anchor point 2222 within the space 2210 between the first pair of plates 2208. Similarly, the second outer element 2218 is coupled to the generally circular central portion 2204 via SSM spring 2224, which is shown in FIGS. 22B and 22C but not in FIG. 22A, and which is anchored to the generally circular central portion 2204 via anchor point 2226 within the space 2214 between the second pair of plates 2212. In FIG. 22A, the two-way SMM springs 2220 and 2224 are in their non-extended states, which causes the first outer element 2216 and the second outer element 2218 to be pulled inwardly toward the generally circular central portion 2204. The dashed-line boxes 2228 and 2230 in FIG. 22A indicate the location of small recesses formed in the end faces of the first outer element 2216 and of the second outer element 2218, respectively, within which the two way SMM springs 2220 and 2224, respectively, are housed when the expandable adjustment block 2200 is in the configuration that is shown in FIG. 22A.

FIG. 22B shows the expandable adjustment block 2200 after the temperature of the two-way SMM springs 2220 and 2224 has been changed and the two-way SMM springs 2220 and 2224 have changed shape from their non-extended state to their extended state. More particularly, the temperature change has caused the two-way SMM springs 2220 and 2224 lengthen and thereby provide the force that is required to push the first outer element 2216 and the second outer element 2218 away from one another and away from the generally circular central portion 2204, resulting in an increase of the size of the expandable adjustment block 2200 along the direction that is indicated by the double-headed arrow in FIG. 22B.

FIG. 22C shows an isometric view of the expandable adjustment block 2200 when the two-way SMM springs 2220 and 2224 are in their extended states. In FIG. 22C, the generally circular central portion 2204 and the first and second pairs of plates 2208 and 2212, respectively, are more clearly visible. Also visible in FIG. 22C is the small recess 2230 that is formed in the end face of the second outer element 2218, within which the two-way SMM spring 2224 is contained when in its non-extended state, as described with reference to FIG. 22A.

Advantageously, the expandable adjustment block 2200 may be used as both the first adjustment block 212 for adjusting the final position of the lower plate 104 relative to the top plate 102, and as the second adjustment block 212a for adjusting the initial position of the lower plate 104 relative to the top plate 102. As is shown in FIGS. 20A through 21B, the first and second adjustment blocks 212 and 212a, respectively, are mounted on the same threaded screw 200, and are disposed between the lower surface 204 of the top plate 102 and the upper surface 210 of the bottom plate 104.

When the two-way SMM springs 2202 of the expandable adjustment block 2200 are in their non-extended state, as shown in FIG. 22A, all of the dimensions of the adjustment block 2200 are sufficiently small to allow the expandable adjustment block 2200 to rotate between the lower surface 204 and the upper surface 210. In this configuration, when the threaded screw 200 is turned, the expandable adjustment block 2200 also turns with the threaded screw and does not advance along the length direction of the threaded screw.

On the other hand, when in the configuration that is shown in FIG. 22B, the expandable adjustment block 2200 is too large to turn with the threaded screw 200 when the threaded screw 200 is rotated. This is because the first outer element 2216 and the second outer element 2218 away from one another and away from the generally circular central portion 2204, resulting in an increase of the size of the expandable adjustment block 2200 along the direction that is indicated by the double-headed arrow. In this configuration, the first outer element 2216 and/or the second outer element 2218 come into contact with the lower surface 204 and/or the upper surface 210, preventing rotation. Rotating the threaded screw 200 now results in relative movement between the threaded screw 200 and the expandable adjustment block 2200, and thus the expandable adjustment block 2200 is forced to move along the length direction of the threaded screw 200. As will be apparent, turning the threaded screw 200 in a clockwise direction will cause the expandable adjustment block 2200 to move in a first direction along the length, and turning the threaded screw 200 in a counter-clockwise direction will cause the expandable adjustment block 2200 to move in a second direction that is opposite the first direction.

As mentioned above, use of a two-way SMM to form the springs 2202 allows the expandable adjustment block 2200 to be used as both the first adjustment block 212 and as the second adjustment block 212a. More particularly, the two-way SMM is programmed differently when it is used in the first adjustment block 212 compared to when it is used in the second adjustment block 212a, such that the “remembered” shape of the two-way SMM springs 2202 is opposite in each of the two blocks 212 and 212a. For instance, the two-way SMM springs 2202 are programmed to have a remembered non-extended shape at room temperature (e.g., about 20° C.) and a remembered extended shape at just below normal human body temperature (e.g., about 35° C.) when being used as the second adjustment block 212 a for adjusting the initial position. On the other hand, the two-way SMM springs 2202 are programmed to have a remembered extended shape at room temperature (e.g., about 20° C.) and a remembered non-extended shape at just below normal human body temperature (e.g., about 35° C.) when being used as the first adjustment block 212 for adjusting the final position.

Adjustment of the initial and final position of the top plate 102 relative to the bottom plate 104 of the oral mandible displacement device may be achieved by firstly cooling the device, or at least the two-way SMM springs 2202 of the first and second adjustment blocks 212 and 212 a, respectively, to about room temperature (e.g., about 20° C.). At this initial temperature, the two-way SMM springs 2202 of the second adjustment block 212a have an extended shape, pushing apart the two halves 2216 and 2218 of the adjustment block as shown in FIG. 22B. One of the dimensions of the second adjustment block 212a is now sufficiently large that it comes into contact with surfaces within the channel through which the threaded screw 200 extends, such that when the threaded screw 200 is turned, the adjustment block 212a is prevented from rotating with the threaded screw 200 and therefore moves along length of the threaded screw 200. In this way, the final position of the top plate 102 relative to the bottom plate 104 may be set.

Next, the device, or at least the two-way SMM springs 2202 of the first and second adjustment blocks 212 and 212a, respectively, is heated to just below normal human body temperature (e.g., about 35° C.). Heating the device to this higher temperature causes the two-way SMM springs 2202 in both the first and second adjustment blocks 212 and 212a to undergo a shape change between their remembered shapes. In the case of the second adjustment block 212a, the SMM springs 2202 undergo a shape change from their extended shape to their non-extended shape, thereby pulling together the two halves 2216 and 2218 of the adjustment block 2200 as shown in FIG. 22A. All of the dimensions of the adjustment block 212a are now sufficiently small that the outer surface of the second adjustment block 212a does not come into contact with any surfaces of the channel through which the threaded screw 200 extends, and thus the adjustment block 212a is able to rotate freely within the channel. Turning the threaded screw 200 does not move the second adjustment block along the length of the threaded screw 200 in this case and therefore does not affect the setting of the final position of the top plate 102 relative to the bottom plate 104.

At the same time, heating the device to this higher temperature also causes the SMM springs 2202 in the first adjustment block 212 to undergo a shape change between their remembered shapes. In the case of the first adjustment block 212, the SMM springs 2202 undergo a shape change from their non-extended shape to their extended shape, thereby pushing apart the two halves 2216 and 2218 of the adjustment block 2200 as shown in FIG. 22B. One of the dimensions of the first adjustment block 212 is now sufficiently large that it comes into contact with surfaces within the channel through which the threaded screw 200 extends, such that when the threaded screw 200 is turned, the first adjustment block 212 is prevented from rotating with the threaded screw 200 and therefore moves along length of the threaded screw 200. In this way, the initial position of the top plate 102 relative to the bottom plate 104 may be set.

Optionally, a not illustrated locking mechanism is provided to prevent unintentional rotation of the threaded screw 200 after the initial and final positions of the top plate 102 relative to the bottom plate 104 have been set, as discussed above.

As discussed previously, oral mandible displacement devices have a top plate with an upper surface for receiving the maxillary dentition of the wearer and a bottom plate with a lower surface for receiving the mandibular dentition of the wearer. The upper surface and the lower surface are each provided with a layer of moldable gel. During a fitting procedure, the moldable gel is softened by heating and the wearer bites down on the gel layers to create an impression therein matching their unique dental structure. After hardening, the impressions formed in the gel layers are precisely matched to the wearer's dental structure, forming a close fit therebetween, which acts to secure the device within the wearer's mouth. One problem associated with the use of oral mandible displacement devices is that the wearer may open their mouth during sleep causing the upper and/or lower surfaces of the device to move away from the wearer's maxillary and or mandibular dentition.

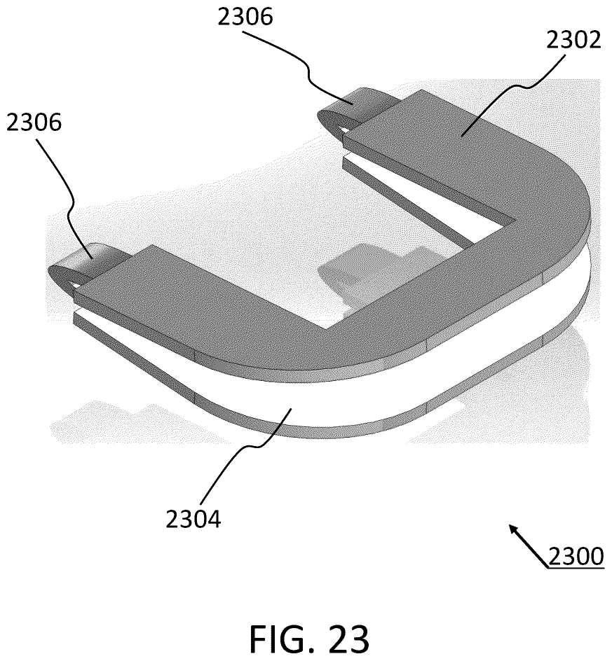

Referring now to FIG. 23, shown is a simplified diagram of a hinged system 2300 for keeping the respective upper and lower surfaces of the top and bottom plates pressed into contact against the wearer's teeth when the wearer's mouth opens during sleep. One end of the top plate 2302 is joined to a corresponding end of the bottom plate 2304 via a pair of hinges 2306. The hinges 2306 may be fabricated using any suitable plastic or metallic material. In one implementation, the hinges normally bias the top plate 2302 away from the bottom plate 2304, providing the force required to maintain contact between the wearer's teeth and the upper and/or lower surfaces of the device 2300. In another implementation, the hinges 2306 are fabricated from a shape memory material that activates upon warming to the activation temperature thereof after heat is transferred from the wearer's mouth to the hinges 2306. Once activated, the SMM hinges provide the force required to maintain contact between the wearer's teeth and the upper and/or lower surfaces of the device 2300. The use of a SMM to fabricate the hinges 2306 provides improved comfort while the wearer is falling asleep.

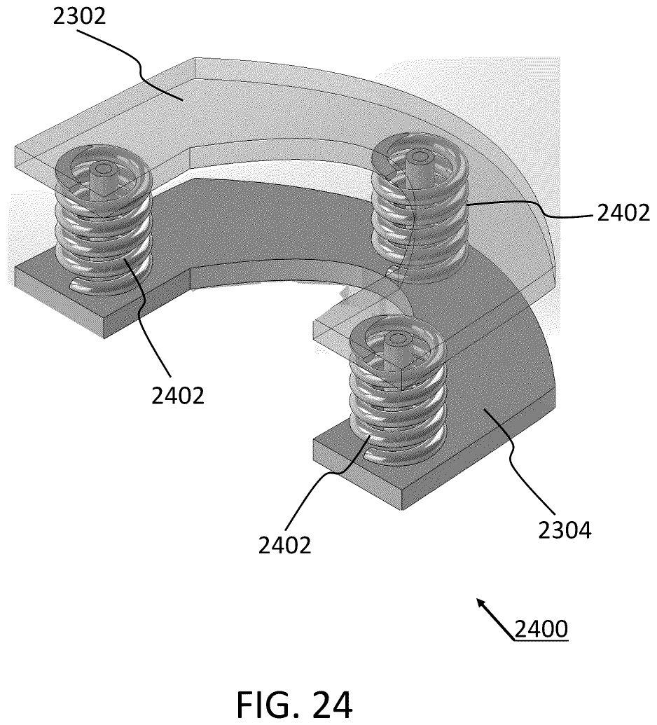

Referring now to FIG. 24, shown is a simplified diagram of another system 2400 for keeping the respective upper and lower surfaces of the top and bottom plates pressed into contact against the wearer's teeth when the wearer's mouth opens during sleep. Instead of joining the top and bottom plates 2302 and 2204 via hinges 2306, the embodiment shown in FIG. 24 uses a plurality of springs 2402 to provide the force required to maintain contact between the wearer's teeth and the upper and/or lower surfaces of the device 2400. In one implementation, the plurality of springs 2402 normally biases the top plate 2302 away from the bottom plate 2304, providing the force required to maintain contact between the wearer's teeth and the upper and/or lower surfaces of the device 2400. In another implementation, the plurality of hinges 2402 are fabricated from a shape memory material that activates upon warming to the activation temperature thereof after heat is transferred from the wearer's mouth to the springs 2402. Once activated, the SMM springs 2402 provide the force required to maintain contact between the wearer's teeth and the upper and/or lower surfaces of the device 2400. The use of a SMM to fabricate the springs 2402 provides improved comfort while the wearer is falling asleep.

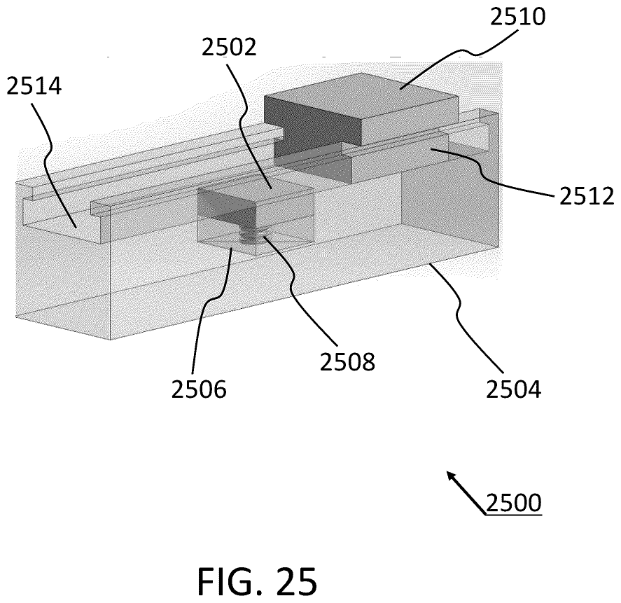

Yet another problem associated with the use of oral mandible displacement devices is that the after the bottom plate has been extended to its final position relative to the top plate, the wearer may inadvertently cause the bottom plate to partially retract. For instance, the wearer may move their jaw, such as during a yawn, during swallowing, etc., causing a motion that pulls the bottom plate away from its desired final position. FIGS. 25-35 show various systems for preventing undesirable movement of the bottom plate away from its desired final position.

Referring now to FIGS. 25-27, shown is a system 2500 including a blocking element 2502 for preventing retraction of bottom plate 2504 away from its desired final position during sleep. The blocking element 2502 is disposed within a recess 2506 formed within the bottom plate 2504.

Now referring specifically to FIG. 25, the blocking element 2502 is formed using a suitable material, such as for instance a plastic material, and is shaped to be able to slide within the recess 2506. A spring 2508 or other type of element for providing a force for displacing the blocking element 2502 is also disposed within the recess 2506. In this specific and non-limiting example, the spring 2508 is formed using a shape memory material with an activation temperature close to normal human body temperature. Upon inserting the device 2500 into the wearer's mouth, heat is transferred to the spring 2508, raising the SMM to its activation temperature, and thereby causing the spring 2508 to change its shape and lengthen. Upon lengthening, the spring 2508 pushes the blocking element 2502 upwards such that it is partially protruding from the recess 2506, and thereby forming a stop that prevents a slider 2510 coupled to the top plate (not shown in FIG. 25) from moving. As is shown in FIG. 25, the slider 2510 has a lower portion 2512 that is shaped to slide within a groove 2514 formed within the bottom plate 2504. The recess 2506 is formed within a portion of the bottom of the groove 2514, and the top surface of the blocking element 2502 is either flush with the bottom of the groove 2514 or is slightly recessed below the bottom of the groove 2514 prior to the spring 2508 activating.

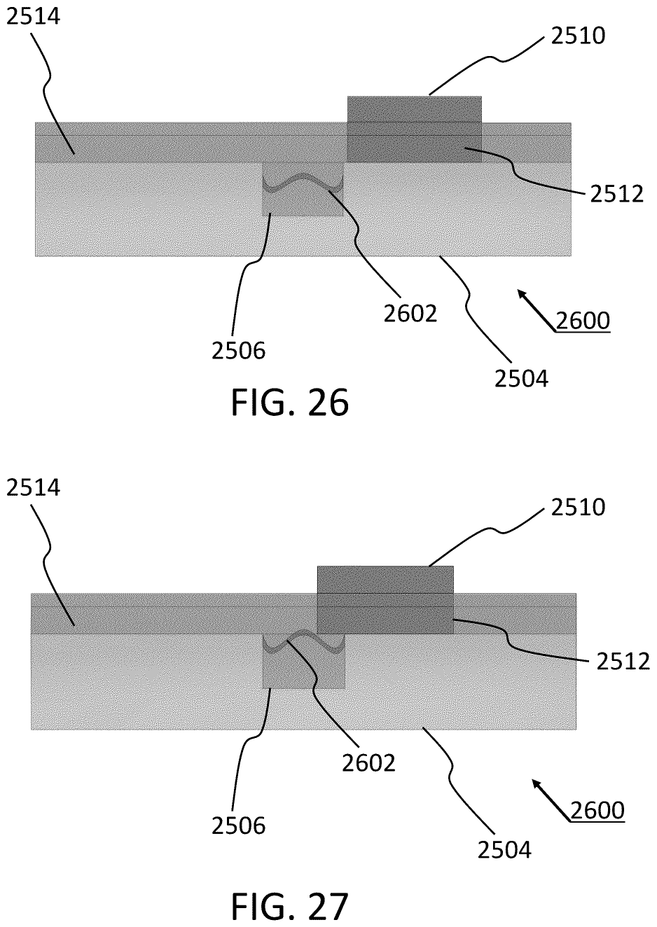

FIG. 26 show another system 2600 for preventing retraction of bottom plate 102 during sleep. In the system 2600, the separate blocking element 2502 and spring 2508 are replaced by a single plate 2602, which is formed from a shape memory material (SMM). More particularly, FIG. 26 shows the plate 2602 when it is at a temperature that is below the activation temperature of the SMM. In the illustrated configuration, the slider 2510 can slide freely within the groove 2514, since the plate 2602 does not protrude out of the recess 2506 that is formed within a portion of the bottom of the groove 2514.

FIG. 27 show the system of FIG. 26 after heating above the activation temperature of the SMM from which the plate 2602 is fabricated. Heating above the activation temperature of the SMM, such as for instance by transferring heat from the wearer's mouth to the plate 2602, causes the plate 2602 to assume a shape in which a portion of the plate 2602 protrudes out of the recess 2506 that is formed within a portion of the bottom of the groove 2514. The protruding portion of the plate 2602 interferes with the lower portion 2512 of the slider 2510, preventing relative movement between the bottom plate 2504 and the not illustrated top plate coupled to the slider 2510.

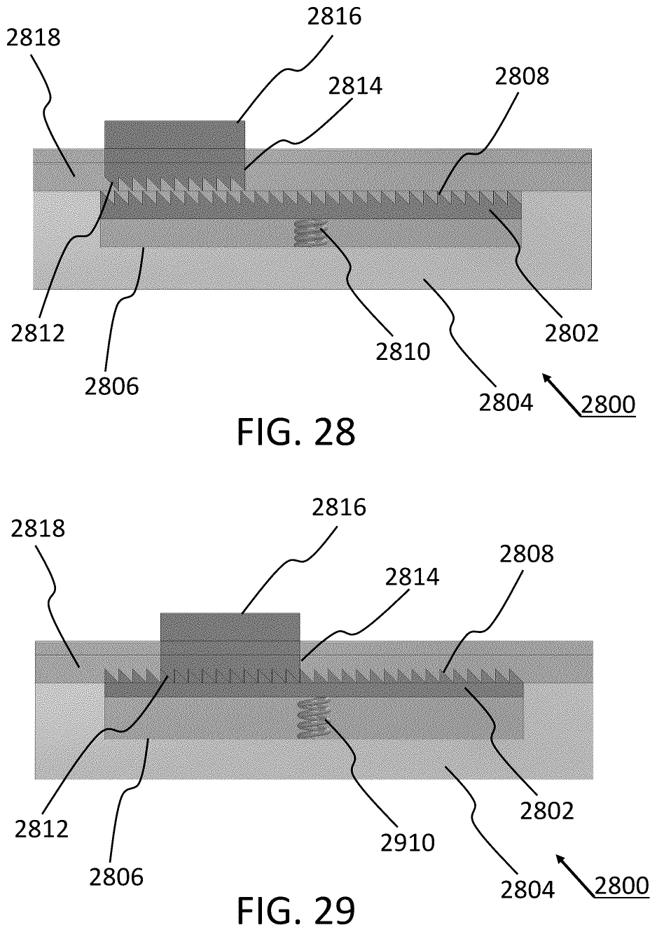

FIG. 28 shows another system 2800 including a blocking element 2802 for preventing retraction of bottom plate away from its desired final position during sleep. The blocking element 2802 is disposed within a recess 2806 formed within bottom plate 2804.

Now referring specifically to FIG. 28, the blocking element 2802 is formed using a suitable material, such as for instance a plastic material, and is shaped to be able to slide within the recess 2806. The blocking element 2802 has a substantially saw-toothed structure 2808 along a top surface thereof. A spring 2810 or other type of element for providing a force for displacing the blocking element 2802 is also disposed within the recess 2806. In this specific and non-limiting example, the spring 2810 is formed using a shape memory material with an activation temperature close to normal human body temperature. Upon inserting the device 2800 into the wearer's mouth, heat is transferred to the spring 2810, raising the SMM to its activation temperature, and thereby causing the spring 2810 to change its shape and lengthen. Upon lengthening, the spring 2810 pushes the blocking element 2802 upwards such that it is partially protruding from the recess 2806 and engages a correspondingly saw-toothed structure 2812 formed along a lower surface of a lower portion 2814 of slider 2816. Since the (not illustrated) top plate is coupled to the slider 2816, when the structure 2808 engages the structure 2812 the slider is prevented from sliding within the groove 2818 and therefore the top plate is prevented from moving relative to the bottom plate 2804. Additionally, by using a spring 2810 of sufficiently low force, the slider 2816 and thus top plate may be allowed to slide only in one direction along the sawtooth pattern, but not against it. In this case, referring to FIGS. 28-29 the slider 2816 could be allowed to slide to the left, but not toward the right.

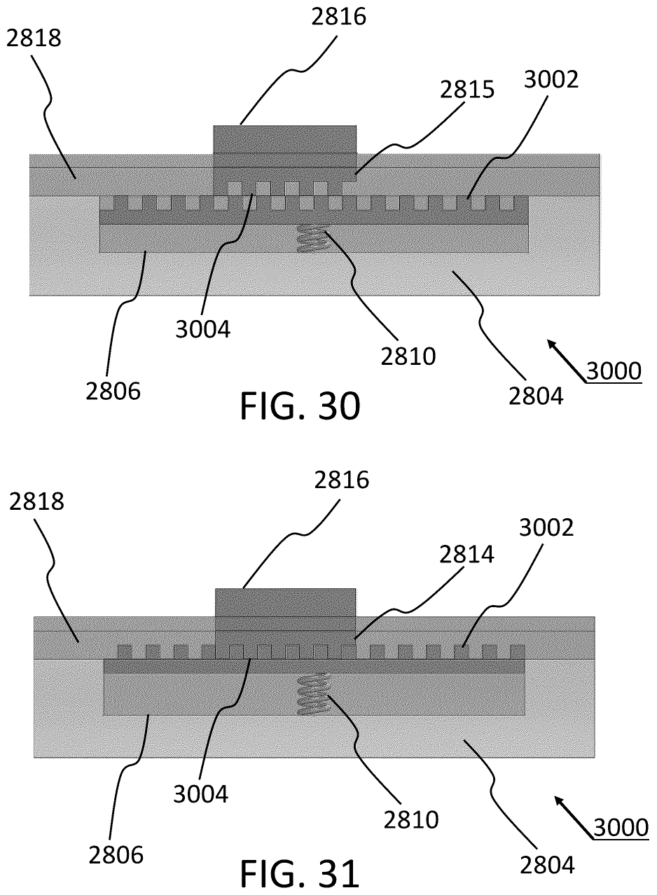

FIG. 30 shows yet another system 3000 for preventing retraction of bottom plate during sleep before activation. The system 3000 is substantially similar to the system 2800, except in system 3000 a square-tooth structure 3002 is formed on the top surface of blocking element 2802 and a corresponding square-tooth structure 3004 is formed on the lower surface of a lower portion 2814 of slider 2816. Since the (not illustrated) top plate is coupled to the slider 2816, when the structure 3002 engages the structure 3004 the slider 2816 is prevented from sliding within the groove 2818 and therefore the top plate is prevented from moving relative to the bottom plate 2804.

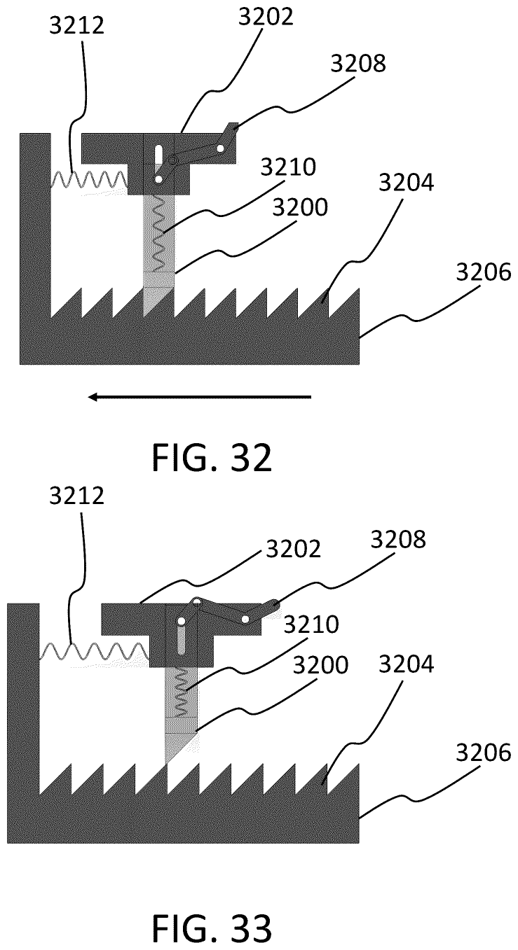

In some embodiments, when the oral mandible displacement device is inserted into the wearer's mouth and the top plate and the bottom plate thereof assume their final positions one relative to another, a mechanism similar to the ones shown in FIGS. 32 through 35 may be used to retain the top and bottom plates in their desired positions during sleep, and then subsequently allow the wearer to easily unlock the device prior to removing the device from the mouth.

FIGS. 32 and 33 show a first implementation comprising a pawl 3200 that is coupled to a portion 3202 of the top plate (not shown in FIGS. 32 and 33). The pawl 3200 can move along a saw-toothed structure 3204 formed on a portion 3206 of the bottom plate (not shown in FIGS. 32 and 33), such as for instance when the bottom plate moves along an extension direction indicated by the arrow in FIG. 32. Once extended, the pawl 3200 prevents the bottom plate from moving back to its original position.

In order to unlock the top and bottom plates and remove the device from the mouth, the wearer may use release lever 3208 to cause the pawl 3200 to disengage the saw-toothed structure 3204. Once the pawl 3200 is retracted from the saw-toothed structure 3204, the bottom plate may be displaced relative to the top plate, which decreases pressure between the teeth and gel of the oral device. Spring 3210 may be provided to normally bias the pawl 3200 in a condition in which it engages the saw-toothed structure 3204. Spring 3412 may provide a force to retract the bottom plate relative to the top plate after the pawl 3200 has been disengaged from the saw-toothed structure 3204.

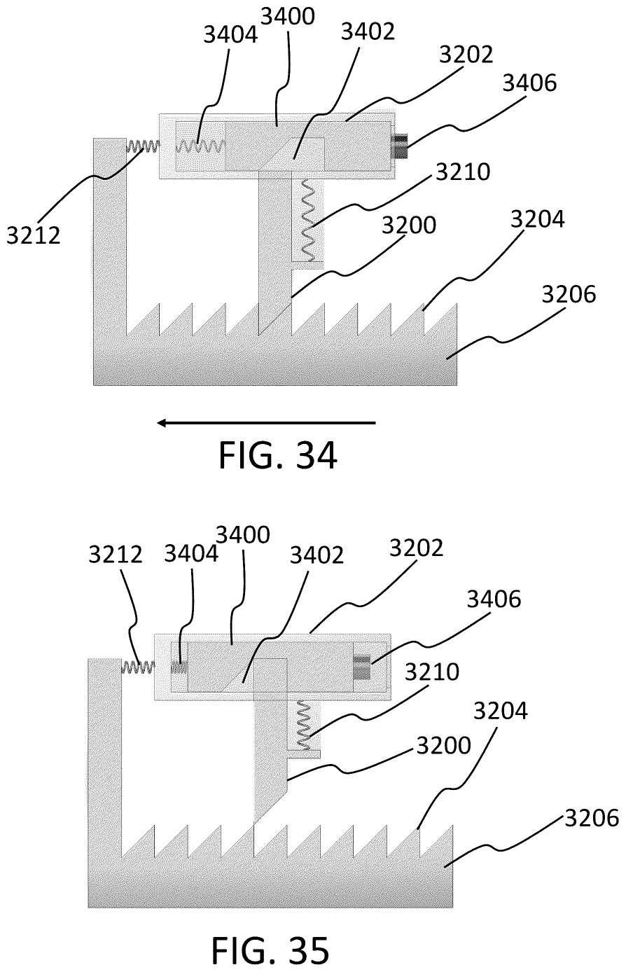

FIGS. 34 and 35 show a second implementation comprising a pawl 3200 that is coupled to a portion 3202 of the top plate (not shown in FIGS. 34 and 35). The pawl 3200 engages a saw-toothed structure 3204 formed on a portion 3206 of the bottom plate (not shown in FIGS. 34 and 35), which prevents the bottom plate from moving back to its original position. The portion 3202 of the top plate comprises a block 3400 with a recess 3402 formed in the lower surface thereof. Spring 3404 normally biases the block 3400 such that the recess 3402 is not aligned with the pawl 3200. In this configuration, the pawl 3200 cannot disengage from the saw-toothed structure 3204.

In order to unlock the top and bottom plates and remove the device from the mouth, the wearer may use release button 3406 to cause the recess 3402 in the block 3400 to move into alignment with the pawl 3200. Spring 3210 in this case normally biases the pawl 3200 away from the saw-toothed structure 3204, and provides the force needed to withdraw the pawl 3200 into the recess 3402 in the block 3200. Once the pawl 3200 is retracted from the saw-toothed structure 3204, the bottom plate may be displaced relative to the top plate, which decreases pressure between the teeth and gel of the oral device. Spring 3412 may provide a force to retract the bottom plate relative to the top plate after the pawl 3200 has been disengaged from the saw-toothed structure 3204.

Yet another problem associated with the use of oral mandible displacement devices is that with frequent use of the device, degradation of the gel teeth impression may significantly impact the device's ability to displace the lower mandible safely. A design which allows for the gel to be easily replaced is proposed.

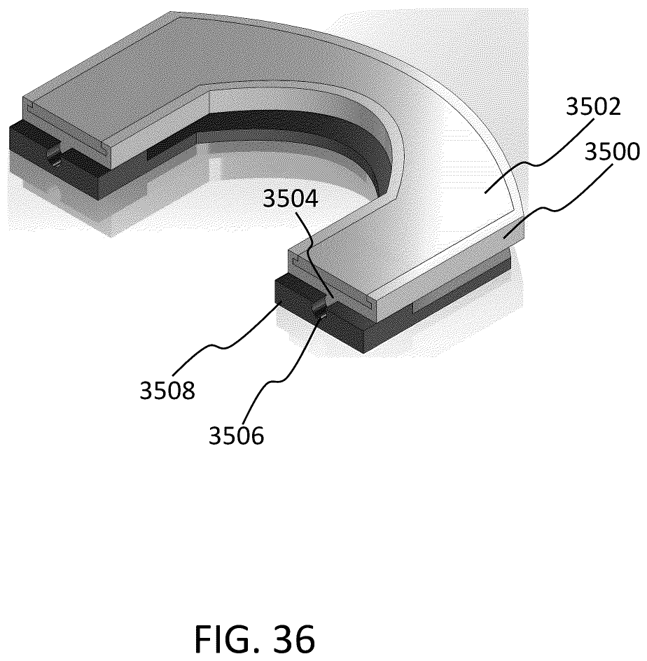

One such design is shown in FIG. 36, which allows for gel mold impression trays 3500 to be interchanged as normal use wears down the integrity of the impression gel 3502. In the example shown in FIG. 36, the impression gel 3502 fits inside impression tray 3500. The impression tray 3500 has a male coupling structure 3504 along a lower surface thereof that is slidably connectable to the female coupling structure 3506 of the top tray 3508. The impression tray 3500 and impression gel 3502 can be replaced as necessary, for instance when the component degrades.

Throughout the description and claims of this specification, the words “comprise,” “including,” “having” and “contain” and variations of the words, for example “comprising” and “comprises” etc., mean “including but not limited to,” and are not intended to, and do not exclude other components.

The terminology used herein is for the purpose of describing particular embodiments only and is not intended to be limiting. As used in the specification and the appended claims, the singular forms “a“ ”and” and “the” include plural references unless the context clearly dictates otherwise.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It will be appreciated that variations to the foregoing embodiments of the disclosure can be made while still falling within the scope of the disclosure. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the disclosure are applicable to all aspects of the disclosure and may be used in any combination. Likewise, features described in non-essential combinations may be used separately (not in combination).

What is claimed is:

1. A configurable time-delayed oral mandible displacement device, comprising:
a top plate configured for engaging maxillary teeth of a user and a bottom plate configured for engaging mandibular teeth of the user, the bottom plate slidably coupled to the top plate for relative movement between a non-extended position and an extended position for displacing a mandible of the user during sleep; and
an adjustment mechanism for selectably setting at least an initial position of the bottom plate relative to the top plate, within a predetermined range of initial positions, the adjustment mechanism comprising at least one element fabricated from a shape memory material (SMM).

2. The configurable time-delayed oral mandible displacement device according to claim 1, wherein the adjustment mechanism includes an anchor element that is configured to be retained within a complementary mating structure disposed within one of the top plate and the bottom plate, and wherein the at least one element fabricated from a shape memory material is a spring extending between the anchor element and the other one of the top plate and the bottom plate.

3. The configurable time-delayed oral mandible displacement device according to claim 2, wherein the anchor element comprises a toothed side surface, wherein the complementary mating structure comprises a groove with a toothed side surface, and wherein during use, teeth formed along the toothed side surface of the anchor element engage teeth formed along the toothed side surface of the groove in a press-fit manner.

4. The configurable time-delayed oral mandible displacement device according to claim 3, wherein the adjustment mechanism supports at least five, discrete initial positions of the bottom plate relative to the top plate.

5. The configurable time-delayed oral mandible displacement device according to claim 3, wherein the spring changes shape when heated to an activation temperature of the SMM, by transfer of body heat from the user to the spring, and wherein the change of shape provides a force that is directed to displace the bottom plate, relative to the top plate, from the initial position to the extended position.

6. The configurable time-delayed oral mandible displacement device according to claim 5, further comprising a lock-element fabricated from a SMM, the lock-element disposed between the top plate and the bottom plate, wherein the lock-element holds the bottom plate in the initial position when a temperature of the lock-element is below an activation temperature thereof, and wherein the lock-element changes shape when heated to its activation temperature by absorbing body heat from the user, said change of shape unlocking the bottom plate from the initial position.

7. The configurable time-delayed oral mandible displacement device according to claim 1, wherein the adjustment mechanism comprises:
a threaded screw having a length;
a first stop member mounted on the threaded screw and being moveable along the length thereof for setting one of the initial position and a final position of the bottom plate relative to the top plate; and
a second stop member mounted on the threaded screw for setting the other one of the initial position and the final position of the top plate relative to the bottom plate;
wherein the first stop member comprises an element fabricated from a shape memory material (SMM) for switching the first stop member between an adjustable condition in which the first stop member is movable along the length of the threaded screw and a non-adjustable condition in which the first stop member is other than moveable along the length of the threaded screw;
wherein the second stop member comprise an element fabricated from a SMM for switching the second stop member between an adjustable condition in which the second stop member is moveable along the length of the threaded screw and a non-adjustable condition in which the second stop member is other than moveable along the length of the threaded screw; and
wherein the element for switching the first element and the element for switching the second element are configured to support movement of the first stop member but not movement of the second stop member along the length of the threaded screw at a first temperature and to support movement of the second stop member but not movement of the first stop member along the length of the threaded screw at a second temperature that is different than the first temperature.

8. The configurable time-delayed oral mandible displacement device according to claim 7, wherein the SMM of the first stop member is a two-way SMM programmed to change shape at two different activation temperatures and wherein the SMM of the second stop member is a two-way SMM programmed to change shape at two different activation temperatures, and wherein the two-way SMM of the first stop member is programmed differently than the two-way SMM of the second stop member.

9. The configurable time-delayed oral mandible displacement device according to claim 7, wherein the element of the first stop member fabricated from a SMM comprises a first pair of springs fabricated from a two-way SMM and wherein the element of the second stop member fabricated from a SMM comprises a second pair of springs fabricated from the two-way SMM.

10. The configurable time-delayed oral mandible displacement device according to claim 9,
wherein:
the first stop member comprises:
a central portion having a threaded through-hole for engaging the thread of the threaded screw;
a first outer element coupled to the central portion via a first spring of the first pair of springs; and
a second outer element coupled to the central portion via a second spring of the first pair of springs, and
wherein:
the second stop member comprises:
a central portion having a threaded through-hole for engaging the thread of the threaded screw;
a first outer element coupled to the central portion via a first spring of the second pair of springs; and
a second outer element coupled to the central portion via a second spring of the second pair of springs.

11. The configurable time-delayed oral mandible displacement device according to claim 10, wherein:
the two-way SMM of the first and second springs of the first pair of springs is programmed to undergo a first change of shape at a first activation temperature and to undergo a second change of shape at a second activation temperature, and
the two-way SMM of the first and second springs of the second pair of springs is programmed to undergo the second change of shape at a third activation temperature and to undergo the first change of shape at a fourth activation temperature.

12. The configurable time-delayed oral mandible displacement device according to claim 11, wherein the first change of shape is substantially opposite the second change of shape.

13. The configurable time-delayed oral mandible displacement device according to claim 12, wherein the first and third activation temperatures are lower than the second and fourth activation temperatures.

14. The configurable time-delayed oral mandible displacement device according to claim 12, wherein the first change of shape is one of a lengthening change and a shortening change, and wherein the second change of shape is the other one of a lengthening change and a shortening change.

15. A configurable time-delayed oral mandible displacement device, comprising:
a top plate configured for engaging maxillary teeth of a user and a bottom plate configured for engaging mandibular teeth of the user, the bottom plate slidably coupled to the top plate for relative movement between a non-extended position and an extended position for displacing the mandible of the user during sleep; and
a screw adjustment mechanism for setting an initial position of the bottom plate relative to the top plate, within a predetermined range of initial positions, and for setting a final position of the bottom plate relative to the top plate, within a predetermined range of final positions, the screw adjustment mechanism comprising:
a threaded screw;
a first stop member comprising a first central portion having a first threaded through-hole for engaging the threaded screw and having first and second outer portions coupled to the first central portion via a first pair of springs fabricated from a two-way shape memory material (SMM), and
a second stop member comprising a second central portion having a second threaded through-hole for engaging the threaded screw and having first and second outer portions coupled to the second central portion via a second pair of springs fabricated from the two-way SMM,
wherein the first pair of springs and the second pair of springs each have a remembered extended shape and a remembered non-extended shape,
wherein the two-way SMM of the first set of springs is programmed such that at a first temperature the first set of springs assume the remembered extended shape and at a second temperature the first set of springs assume the remembered non-extended shape, and
wherein the two-way SMM of the second set of springs is programmed such that at the first temperature the second set of springs assume the remembered non-extended shape and at the second temperature the second set of springs assume the remembered extended shape.

16. The configurable time-delayed oral mandible displacement device according to claim 15, wherein the first set of springs push the first and second outer portions of the first stop member away from the first central portion at the first temperature such that at least one of the first and second outer portions contacts a surface adjacent to the threaded screw and thereby prevents the first stop member from turning with the threaded screw, such that the first stop member moves along the threaded screw when the threaded screw is turned and wherein the second set of springs pulls the first and second outer portions of the second stop member toward the second central portion at the first temperature, such that the first and second outer portions of the second stop member other than contact the surface.

17. The configurable time-delayed oral mandible displacement device according to claim 16, wherein the first set of springs pulls the first and second outer portions of the first stop member toward the first central portion at the second temperature such that the first and second outer portions other than contact the surface, and wherein the second set of springs push the first and second outer portions of the second stop member away from the second central portion at the second temperature such that at least one of the first and second outer portions contacts the surface adjacent to the threaded screw and thereby prevents the second stop member from turning with the threaded screw such that the second stop member moves along the threaded screw when the threaded screw is turned.

18. The configurable time-delayed oral mandible displacement device according to claim 17, wherein the first stop member is for setting the initial position of the bottom plate relative to the top plate when the device is at the first temperature.

19. The configurable time-delayed oral mandible displacement device according to claim 17, wherein the second stop member is for setting the final position of the bottom plate relative to the top plate the device is at the second temperature.
